(19) United States
(12) Patent Application Publication
(10) Pub. No.: US 2025/0022603 A1
(43) Pub. Date: Jan. 16, 2025

(54) PREDICTING PROGRESSION OF COGNITIVE IMPAIRMENT

(71) Applicant: EISAI R&D MANAGEMENT CO., LTD., Tokyo (JP)

(72) Inventors: Viswanath DEVANARAYAN, Souderton, PA (US); Shobha DHADDA, Upper Saddle River, NJ (US); Michael IRIZARRY, Tenefly, NJ (US)

(21) Appl. No.: 18/523,799

(22) Filed: Nov. 29, 2023

Related U.S. Application Data
(60) Provisional application No. 63/593,433, filed on Oct. 26, 2023.
(60) Provisional application No. 63/513,799, filed on Jul. 14, 2023.

(51) Int. Cl. G16H 50/30; G06N 20/00; G16B 20/00; G16B 25/10

(52) U.S. Cl. CPC G16H 50/30; G06N 20/00; G16B 20/00; G16B 25/10

(57) ABSTRACT

A platform is disclosed for the training and deployment of statistical or machine-learning models for predicting the progression of cognitive impairment or brain amyloid status. The statistical or machine-learning models can be trained to predict the progression of cognitive impairment or brain amyloid status using baseline image data, biomarker data, genomic data, demographic data, cognitive data, or the like. The platform can be configured to obtain training data, train the statistical or machine-learning models, and support using the trained statistical or machine-learning models to respond to prediction requests.

### CROSS-REFERENCE TO RELATED APPLICATIONS

This application claims the benefit of U.S. Provisional Application No. 63/513,799, filed Jul. 14, 2023, and U.S. Provisional Application No. 63/593,433, filed Oct. 26, 2023. The provisional applications identified above are incorporated here by reference in their entireties.

### TECHNICAL FIELD

The present disclosure relates to training machine learning or statistical models to predict progression of cognitive impairment or brain amyloid status for individual subjects.

### BACKGROUND

Patients with neurological disease, dysfunction, or injury can exhibit great variations in progression of cognitive impairment. These variations can depend on their baseline clinical and biological characteristics. This variance in the progression of cognitive impairment can limit the ability of physicians, caregivers, and subjects to make appropriate decisions and plans on treatment and long-term care. Furthermore, such variance can increase the required number of subjects in control and treatment groups in clinical trials of treatments for such neurological diseases, dysfunctions, or injuries. Apart from increasing the difficulty of such trials, increased control group requirements can result in denying patients the benefits of a treatment later proven to be effective.

Conventional methods of detecting brain amyloid status in a patient can require administration of a radioactive tracer to the subject and subsequent collection of imaging data (e.g., performing a PET scan). These significant requirements can prevent widespread screening of patients for brain amyloid status.

### SUMMARY

Systems and methods are disclosed for training predictive models to predict the progression of cognitive impairment or brain amyloid status of subjects. The predictive models can be trained using control data from clinical trials. Predictions of the progression of cognitive impairment or brain amyloid status of a subject can be used for managing care of the subject, for patient selection and enrichment, or as a prognostic covariate in a future clinical trial.

Disclosed embodiments include a system including at least one processor and at least one computer-readable, non-transitory medium containing instructions. The instructions, when executed by the at least one processor, can cause the system to perform operations. The operations can include obtaining training data for first subjects satisfying a cognitive impairment condition. The training data can include, for each first subject, baseline data and cognitive impairment progression data. Baseline data for a first subject can include baseline cognitive data and image data. The imaging data can include one or more measurements for one or more brain regions identified as hubs or one or more composite values for one or more clusters of brain regions identified as structural brain network modules, the hubs or modules identified using network analysis or multi-level clustering. Cognitive impairment progression data for a first subject can include repeated measurements acquired over time, the repeated measurements acquired after the baseline cognitive data. The operations can further include training a machine learning model, using the training data, to predict cognitive impairment progression data for the first subject using baseline data for the first subject. The operations can further include obtaining the baseline data for a second subject, the subject satisfying the cognitive impairment condition. The operations can further include predicting cognitive impairment progression data for the second subject by inputting the baseline data for the second subject to the trained predictive model.

Disclosed embodiments include a system including at least one processor and at least one computer-readable, non-transitory medium containing instructions. The instructions, when executed by the at least one processor, can cause the system to perform operations. The operations can include obtaining training data for first subjects satisfying a cognitive impairment condition. The training data can include, for each first subject, baseline data and cognitive impairment progression data. The baseline data for the first subject can include plasma fluid biomarker data. The operations can further include training a predictive model, using the training data, to predict cognitive impairment progression data for the first subject using baseline data for the first subject. The operations can further include obtaining baseline data for the second subject, the second subject satisfying the cognitive impairment condition. The operations can further include predicting cognitive impairment progression data for the second subject by inputting the baseline data for the second subject to the trained predictive model.

Disclosed embodiments include a system including at least one processor; and at least one computer-readable, non-transitory medium containing instructions. The instructions, when executed by the at least one processor, can cause the system to perform operations. The operations can include obtaining training data for first subjects satisfying a cognitive impairment condition. The training data can include, for each first subject, baseline data and brain amyloid data. The baseline data can include plasma fluid biomarker data. The operations can further include training a predictive model, using the training data, to predict brain amyloid status for the first subject using baseline data for the first subject. The operations can further include obtaining baseline data for a second subject, the second subject satisfying the cognitive impairment condition. The operations can further include predicting brain amyloid status for the second subject by inputting the baseline data for the second subject to the trained predictive model.

The disclosed embodiments further include computer-readable, non-transitory media containing instructions for configuring systems to perform the above-recited operations, and methods corresponding to the above-recited operations.

The foregoing general description and the following detailed description are exemplary and explanatory only and are not restrictive of the claims.

### BRIEF DESCRIPTION OF THE DRAWINGS

The accompanying drawings, which are incorporated in and constitute part of this disclosure, together with the description, illustrate and serve to explain the principles of various example embodiments.

FIGS. 1A to 1D depict the variability in individual progression of Alzheimer's disease.

FIG. 2 depicts an exemplary platform for developing, validating, and deploying predictive models for predicting a progression of cognitive impairment or brain amyloid status, consistent with disclosed embodiments.

FIG. 3 depicts an exemplary process of predicting the progression of cognitive decline in a subject, consistent with disclosed embodiments.

FIG. 4 depicts an exemplary process for identifying hubs and modules in the brain based on regional measurements, consistent with disclosed embodiments.

FIG. 5 depicts an exemplary process for predicting brain amyloid status of a subject, consistent with disclosed embodiments.

FIGS. 6A to 6H concern an investigation into the prediction of cognitive impairment progression and disease progression in amyloid positive subjects with mild cognitive impairment.

FIGS. 7A to 7I concern an investigation into prediction of brain amyloid-β status using blood-based tests.

FIGS. 8A to 8Q concern an investigation into predicting the progression of cognitive impairment.

FIGS. 9A to 9M concern an investigation into predicting the progression of cognitive impairment in early Alzheimer's disease.

FIGS. 10A to 10AA concern an investigation into predicting progression of cognitive impairment in early Alzheimer's disease.

### DETAILED DESCRIPTION

The following detailed description refers to the accompanying drawings. Wherever possible, the same reference numbers are used in the drawings and the following description to refer to the same or similar parts. While several illustrative embodiments are described herein, modifications, adaptations, and other implementations are possible. For example, substitutions, additions, or modifications may be made to the components illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope is defined by the appended claims.

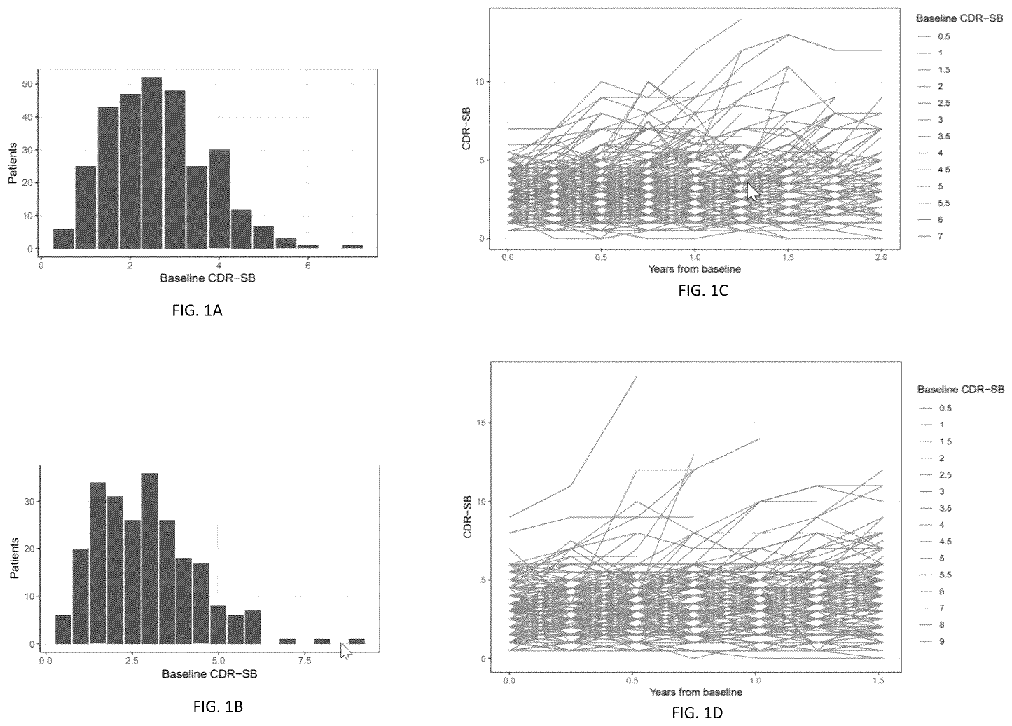

Individual disease progression trajectories can vary greatly between subjects with neurological disease, dysfunction, or injury, depending on their baseline clinical and biological characteristics. FIGS. 1A and 1B depict the distribution of baseline cognitive measurements of individual subjects in a first clinical trial and a second clinical trial respectively, and FIGS. 1C and 1D depict repeated cognitive measurements over time for individual placebo arm subjects of the first and second clinical trials, respectively. As apparent from these figures, which concern subjects with early Alzheimer's Disease (AD), the progression in subject cognitive impairment varies greatly among subjects. This variance in subject cognitive impairment can limit the ability of physicians, caregivers, and subjects to make appropriate decisions and plans on treatment and long-term care.

The disclosed embodiments include predictive models suitable for predicting progression of cognitive impairment in AD subjects. The predictive models can be configured to accept as inputs demographic data, cognitive data (e.g., one or more cognitive measurements), genomic data, imaging data, biomarker data, or other suitable baseline data. The predictive models can provide as outputs predicted cognitive assessment scores (e.g., at predetermined post-baseline interval(s), at post-baseline interval(s) specified in a request, or any other suitable post-baseline time(s)). Trained predictive models consistent with disclosed embodiments can enable physicians, caregivers, and subjects to make appropriate decisions and plans on treatment and long-term care. Furthermore, the predictive model can be used to select appropriate patients for clinical trials or generate prognostic covariate data suitable for use in clinical trials.

The disclosed embodiments further include predictive models suitable for predicting brain Aβ detection probabilities based on blood biomarkers. Such predictive models can be used as a screening tool for detecting brain amyloid burden (e.g., as part of screening for clinical trials or for managing patient care).

The disclosed predictive models can be trained using historical placebo subject data from clinical trials. As appreciated by the inventors, clinical trials provide a particularly powerful set of training data because subjects were assessed at multiple timepoints and were screened prior to study enrollment. The screening restricted the subjects to those likely having early-stage AD-related cognitive impairment. By restricting the training data to subjects having similar etiologies and stage of AD progression, the predictive power of the predictive models can be improved. Furthermore, brain imaging data and biomarker data are available for a substantial proportion of these subjects, providing another source of input data for predicting cognitive impairment progression. In some embodiments, the disclosed predictive models can be trained using other datasets (e.g., research datasets).

Neurological disease, dysfunction, or injury can include any condition that affects the central nervous system, resulting in impaired movement, cognition, or behavior. Neurological disease, dysfunction, or injury can include diseases of the central nervous system (e.g., Alzheimer's disease, dementia, or the like), neurological disorders (e.g., mild cognitive impairment (MCI), or the like), or injuries (e.g., strokes, traumatic brain injury, or the like).

Predictive models can include statistical and machine learning models suitable for identifying relationships between input data and output results. Such models can include regression models (e.g., logistic regression models; ridge, lasso, or elastic net regression models; time series regression models; or the like), support vector machines, Bayes classifiers, neural networks, decision trees, random forests, ensemble models, or other suitable statistical and machine learning models.

In some embodiments, suitable predictive models can include regularized logistic regression models and ensemble tree-based models. Regularized logistic regression models can include Bayesian elastic net models. Such models can use a mixture double-exponential prior to reduce the complexity of the model, thus preventing overfitting and increasing model robustness. Ensemble tree-based models can include Stochastic Gradient Boosting models, which can combine predictions from multiple decision trees to generate the final predictions. The nodes in each of the multiple decision trees can be trained using different random subsets of input features. The individual decision trees can therefore differ and potentially capture different signals from the data.

A predictive model can be trained using a training cohort of subjects having neurological disease, dysfunction, or injury. Neurological disease can include diseases of the central nervous system, such as Alzheimer's disease or dementia. Neurological disorders can include mild cognitive impairment (MCI), or the like. Neurological injury can include strokes, traumatic brain injury, or the like.

The training cohort can include subjects satisfying a screening criterion. The screening criterion can be intended to restrict the training cohort to subjects having the same neurological disease, dysfunction, or injury (or a suitable combination of diseases, dysfunctions, or injuries). For example, as described herein, the subjects may be screened for mild cognitive impairment, or brain amyloid burden. By restricting the training cohort to similarly situated subjects, the performance of the predictive model can be improved.

A predictive model can be trained to predict cognitive impairment progression for an individual subject using input data for that individual subject. The input data can be acquired at or by a baseline date. When different portions of the input data are acquired on different dates, one of these dates may be selected as the baseline date (e.g., the latest of the dates, the date corresponding to the most time-sensitive or varying component of the input data, the date of acquisition of the cognitive measures, the date of acquisition of imaging data, the date of acquisition of biomarker data, some imputed date based on the dates two or more components of the input data were acquired, such as imaging and cognitive date, or another suitable selection for baseline date). Demographic information may be acquired at or prior to the baseline date. The predictive model can output a predicted score for an assessment of cognitive and functional abilities (e.g., at predetermined post-baseline interval(s), at post-baseline interval(s) specified in a request, or any other suitable post-baseline time(s)), such as a Clinical Dementia Rating Sum of Boxes (CDR-SB) score, or other assessments described herein.

The predicted score can be absolute or relative. For example, the predicted score can be the predicted CDR-SB for the subject, or the predicted change in CDR-SB score from a baseline CDR-SB value.

The predictive model can be trained to predict a progression of cognitive impairment for an individual patient. In some embodiments, the predictive model can be configured to output a sequence of predicted scores. For example, given baseline input data, the predictive model can be configured to output predicted scores at 3, 6, 9, 12, 15, 18, 21, 24, 27, 30, 33, 36, 48, or 60 months. In some embodiments, the input data to the predictive model can include a duration, or elapsed time. The predictive model can be configured to output a predicted score for that duration or elapsed time. As may be appreciated, such a model may be capable of outputting different predicted scores for different durations or elapsed times. For example, given the same baseline input data, the predictive model may predict a greater cognitive decline at 18 months than 3 months.

The predictive model can be trained and configured to predict a progression of cognitive impairment for a subject using baseline data for the subject. The baseline data can include baseline cognitive data. The baseline cognitive data can be obtained from the subject, a clinician observing or treating the subject, or another person familiar with the subject.

The baseline cognitive data can include one or more cognitive measurements acquired using one or more cognitive assessments, such as the CDR-SB assessment, Cogstate Brief Battery assessment, the International Shopping List Test assessment, the Alzheimer's Disease Assessment Scale (ADAS) assessment, Alzheimer's Disease Composite Score (ADCOMS) assessment, the mini-mental state examination, the Functional Activities Questionnaire (FAQ), or other suitable cognitive assessments.

As may be appreciated, a cognitive assessment can include multiple components. The Cogstate Brief Battery assessment can include four components that measure different aspects of cognitive function: Detection, Identification, One-Card Learning, and One-Back. The CDR-SB assessment can include six domains: Memory (CDR0101), Orientation (CDR0102), Judgment and Problem Solving (CDR0103), Community Affairs (CDR0104), Home and Hobbies (CDR0105), and Personal Care (CDR0106). The ADAS-13 assessment can include the components: Word Recall (ADCRL), Commands (ADCCMD), Constructional Praxis (ADCCPS), Delayed Word Recall (ADCDRL), Naming (ADCOF), Ideational Praxis (ADCIP), Orientation (ADCOR), Word Recognition (ADCRG), Remembering Test Instructions (ADCRI), Comprehension of Spoken Language (ADCCMP), Word Finding Difficulty (ADCDIF), Spoken Language Ability (ADCSL), and Number Cancellation (ADCNC). The ADCOMS assessment can include memory, language, orientation, executive function, mental processing speed, visuospatial ability, and global functioning components. The Functional Activities Questionnaire can include ten questions concerning activities of daily living: paying bills (FAQ01), assembling records (FAQ02), shopping alone (FAQ03), playing games (FAQ04), heating water and turning off stove (FAQ05), preparing balanced meal (FAQ06), tracking current events (FAQ07), paying attention (FAQ08), remembering appointments (FAQ09), traveling (FAQ10). As may be appreciated, each of these components can be associated with a score. The overall score for an assessment can be a composite of these component-level scores. In some embodiments, the baseline cognitive data can include the composite score for an assessment or score(s) for one or more components of the assessment.

The disclosed embodiments are not limited to any particular version of the above cognitive assessments. For example, the baseline cognitive data can include an ADAS-13 score or an ADAS-14 score. The ADAS-14 questionnaire can include additional items addressing executive function that are not specifically included in the ADAS-13 questionnaire. Similarly, the baseline cognitive data can include FAQ IV scores, or scores for earlier versions of the FAQ questionnaire.

In some embodiments, the baseline data can include demographic data for the subject. The demographic data can include age, sex, weight, body mass index (BMI), or other suitable demographic data.

In some embodiments, the baseline data can include genomic data for the subject. Genomic data can include variant information (e.g., presence or absence of a variant; characteristics of a variant such as deletion size, insert size, frame shift information, copy number, single nucleotide polymorphism information; or the like) for gene variants associated with neurological disease, dysfunction, or injury. For example, genomic data can include apolipoprotein E (APOE) variant data, amyloid protein precursor (APP) variant data, presenilin-1 (PSEN1) variant data, presenilin-2 (PSEN2) variant data, clusterin (CLU) variant data, Triggering Receptor Expressed in Myeloid Cells 2 (TREM2), or the like. Genomic data can further include allelic count information for such variants in a subject.

In some embodiments, the baseline data can include image data of the subject. The image data can be, include, or depend upon features extracted from images of the brain of the subject (or a portion thereof). The brain images can be acquired using magnetic resonance imaging (MRI), computed tomography (CT) imaging, positron emission tomography (PET) imaging, or another suitable modality. In some embodiments, the image data can include measurements of brain regions, whole brain volume, hippocampal volume, or the like. In some embodiments, the measurements can include volume, surface area, or cortical thickness measurements. The volume, surface area, or thickness measurements can be normalized (e.g., using whole brain volume, or another suitable normalization factor). The brain region measurements can be extracted from MRI images, CT images, PET images, or images acquired using another suitable imaging modality. In some embodiments, the image data can include amyloid or tau PET data (e.g., whether detected amyloid or tau satisfies a threshold condition in the brain of the subject or a portion thereof). In some embodiments, the image data can include diagnostic tracer (fluorodeoxyglucose, florbetaben, florbetapir, flutemetamol, or the like) PET data. In some embodiments, the amyloid, tau, or tracer PET data can be expressed in terms of a score (e.g., a normalized or percentile score, or the like).

As described herein, the baseline data can include image data specific to identified hubs and modules in the brain. The hubs and modules can be identified using a data analysis or feature extraction technique, such as network analysis or multi-level clustering. Network analysis can be used to identify suitable brain regions based on connections between these brain regions. Such a network analysis can identify brain regions as being important based on the centrality of the brain region in a network of brain regions (or subnetwork within the overall network), the degree to which the brain region bridges different subnetworks within the network, the degree to which the brain region is connected to other important brain regions, or based on other suitable criteria.

Multi-level clustering can involve repeatedly clustering brain regions together based on similarities in measurements for the brain regions. In each repeat, more (or potentially more) clusters can be generated using smaller subsets of the brain regions. In some embodiments, hierarchical clustering can be performed. A first round of clustering can be performed on the brain regions to generate a first set of clusters. Additional rounds of clustering can be performed on the brain regions within each cluster in the first set of clusters.

In some embodiments, network analysis and multi-level clustering can be combined. For example, hubs and modules can be identified using multiscale embedded gene co-expression network analysis (MEGENA), recursive feature elimination, or the like. Image data specific to identified hubs and modules in the brain can include measurements for hubs (e.g., volume, surface area, cortical thickness, or the like) and composite values for modules (e.g., composite values derived from measurements for the brain regions comprising the modules).

In some embodiments, the baseline data can include biomarker data for the subject. A biomarker can be a measurable substance or characteristic indicative of a biological process or condition, such as a disease state or response to therapy. Biomarker data can include amyloid beta biomarker data, tau biomarker data, neurofilament light peptide (NfL) biomarker data, glial fibrillary acidic protein (GFAP) biomarker data, or the like. For example, biomarker data can include total tau levels, microtubule binding region (MBTR)-tau levels, phosphorylated tau levels (e.g., tau phosphorylated at 181 (p-Tau181) levels, tau phosphorylated at 217 (p-Tau217) levels, tau phosphorylated at 231 (p-Tau231) levels, or the like), neurogranin levels, Aβ1-42 levels, Aβ1-40 levels, or the like. Such biomarker data can be measured in blood (e.g., from plasma, serum, or the like), cerebrospinal fluid, or other suitable bodily fluids of the subject. For example, the baseline data can include serum or plasma Aβ1-42 level, cerebrospinal fluid Aβ1-42 level, or Aβ1-42 level as measured in another suitable bodily fluid.

The biomarker data can include indications of the presence or absence of a biomarker in a sample obtained from the subject, the amount or concentration of the biomarker in the sample, or the like. The biomarker data can be expressed as a measured amount or transformed into a score (e.g., a normalized amount, a percentile, or the like). In some embodiments, the biomarker data can include functions of multiple biomarkers. For example, the biomarker data can include the combination of Aβ1-42 and Aβ1-40 levels (or scores), the ratio of Aβ1-42 to Aβ1-40 levels (or scores), the ratio of p-Tau181 (or p-Tau217 or p-Tau231) to Aβ1-42 (or Aβ1-40) levels (or scores), or the like.

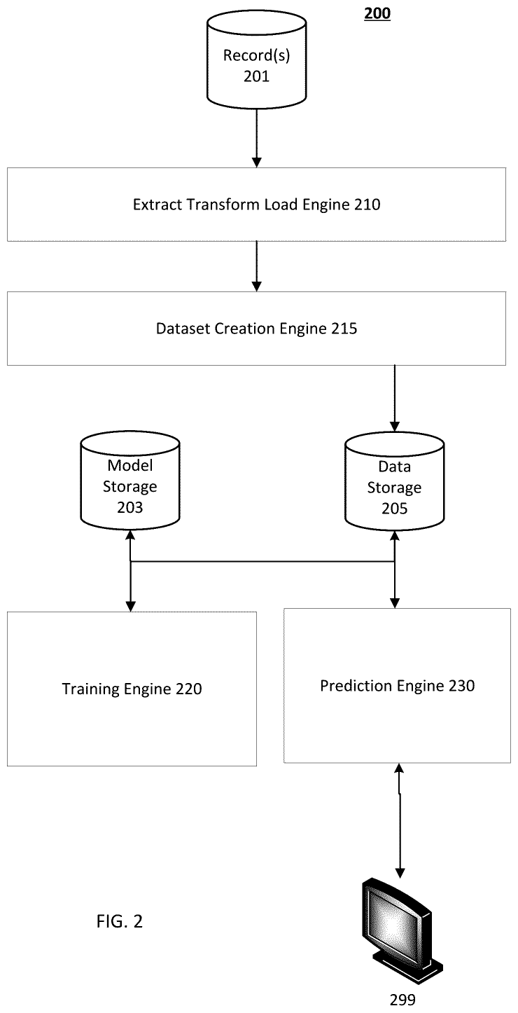

FIG. 2 depicts an exemplary platform 200 for developing, validating, and deploying predictive models for predicting a progression of cognitive impairment or brain amyloid status, consistent with disclosed embodiments. Platform 200 can be configured to obtain input data from other systems (e.g., such as imaging systems or medical laboratory systems, not shown in FIG. 2) or record(s) 201. Consistent with disclosed embodiments, such data can include subject image data, biomarker data, genomic data, demographic data, cognitive data, or the like. Platform 200 can be configured to generate datasets suitable for training predictive models using components such as extract transform load (ETL) engine 210 and dataset creation engine 215. Platform 200 can be configured to train models using training engine 220. Trained models can be used in the prediction phase by prediction engine 230. A user can interact with user device 299 to control and configure platform 200. The user can also provide subject data to, and receive predictions from, platform 200 by interacting with user device 299.

As may be appreciated, the particular arrangement of components depicted in FIG. 2 is not intended to be limiting. Platform 200 can include additional components (e.g., additional databases, data sources, processing systems, or the like) or fewer components (e.g., by combining databases or processing systems). The functionality of the existing components can be combined or distributed among additional systems, without departing from the envisioned embodiments.

Components of FIG. 2 can be implemented using one or more computing systems (e.g., a laptop, desktop, workstation, computing cluster, on-premises or off-premises cloud computing platform, or the like). For example, a computing cluster or workstation can implement ETL engine 210, dataset creation engine 215, or training engine 220. As an additional example, a desktop or laptop (e.g., user device 299 or another device) can implement prediction engine 230. As an additional example, the components of platform 200 (e.g., apart from user device 299) can be implemented using containerized services on a cloud computing platform. As may be appreciated, these examples are not intended to be limiting.

Consistent with disclosed embodiments, record(s) 201 can include one or more storage locations for data usable by platform 200 to predict a progression of cognitive impairment or the brain amyloid status. In some embodiments, such data can include raw or processed image data of subjects. The image data can be MRI images, PET images, CT images, or the like. In various embodiments such data can include medical record information for the subjects. Such medical record information can include medical records, case notes, clinical trial records, requisition information (e.g., pertaining to biomarker testing), or the results of laboratory tests. In some embodiments, the medical record information can include cognitive data usable for constructing training datasets (e.g., cognitive measurements acquired at 3, 6, 9, 12, 15, and 18-month assessments, or other intervals consistent with disclosed embodiments).

Consistent with disclosed embodiments, ETL engine 210 can be configured to obtain data in varying formats from one or more sources (e.g., record(s) 201, or the like). The disclosed embodiments are not limited to any particular format of the obtained data, or method for obtaining this data. For example, the obtained data can be or include structured data or unstructured data. ETL engine 210 can interact with the various data sources to receive or retrieve the data.

ETL engine 210 can transform the data into suitable format(s) and load the transformed data into a target component or database of platform 200. In some embodiments, transforming the data can include performing quality control processing on obtained data. Such quality control processing can include confirming that data is usable (e.g., that the subject satisfies inclusion criteria for the model to be trained, that required input data for a subject is complete, or the like). In some embodiments, transforming the data can include processing the data into a standard format or structure. As may be appreciated, the input data obtained from record(s) 201 may not be in a suitable format for training a predictive model. Similarly, input data obtained from different ones of record(s) 201 may have different formats. Accordingly, ETL engine 210 can clean the obtained input data such that the input data, although originating from a variety of different sources, has a consistent format.

In some embodiments, ETL engine 210 can enrich image data or medical record information by generating additional data using the image data or medical record information. For example, ETL engine 210 can convert biomarker levels to scores (e.g., using population distribution information, clinical ranges, or the like), normalize image data (e.g., normalize area and volume by the intra cranial volume, or the like), or the like. In some embodiments, ETL engine 210 can remove unnecessary or unwanted variables or data from the input dataset. For example, when a medical record contains information unrelated to predicting a progression of cognitive impairment, ETL engine 210 can create a version of the medical record that contains only the information related to predicting cognitive impairment.

Consistent with disclosed embodiments, ETL engine 210 can load the transformed data into another component of platform 200, such as dataset creation engine 215 (or into a suitable data storage, from which dataset creation engine 215 can retrieve the data).

In some embodiments, dataset creation engine 215 can be configured to generate training samples or inference samples from data received from ETL engine 210. Dataset creation engine 215 can be configured to extract any necessary input data features from the transformed data received from ETL engine 210. In some embodiments, dataset creation engine 215 can generate features based on combinations of biomarkers (e.g., ratio of Aβ1-42 to Aβ1-40 score, or the like), determine correlations between input data (e.g., between thickness, area, or volume measurements for different brain regions, or the like), identify brain regions as modules or hubs, as described herein, or perform other feature extraction.

In some embodiments, dataset creation engine 215 can be configured to accept label information provided by a user through user device 299. For example, dataset creation engine 215 can be configured to provide data (or metadata concerning the data) received from ETL engine 210 to user device 299 for display. In response, dataset creation engine 215 can receive label information (e.g., identification of a subject as having brain amyloid, cognitive measurements for a patient obtained during an assessment, or the like).

In some embodiments, dataset creation engine 215 can be configured to associate labels with training samples. For example, when predicting progression of cognitive impairment, the data can include repeated cognitive measurements acquired over time. These repeated cognitive measurements may be acquired after the acquisition of the baseline cognitive data. The dataset creation engine 215 can associate this cognitive impairment progression data with the baseline input data. A training sample can then include the baseline data for the subject and the associated cognitive impairment progression data. As an additional example, when predicting brain amyloid status, a finding of brain amyloid presence can be noted in a medical record of a subject (e.g., based on a visual radiotracer read in PET image data). A training example can then include the baseline data for the subject and the indication of the finding of brain amyloid presence. Dataset creation engine 215 can be configured to store training samples in data storage 205.

Consistent with disclosed embodiments, model storage 203 can be a storage location for models usable by components of platform 200 (e.g., training engine 220, or prediction engine 230). The disclosed embodiments are not limited to any particular implementation of model storage 203. Consistent with disclosed embodiments, model storage 203 can be implemented using one or more relational databases, object-oriented or document-oriented databases, tabular data stores, graph databases, distributed file systems, or other suitable data storage options.

Consistent with disclosed embodiments, data storage 205 can be a storage location for prepared datasets usable by training engine 220 or prediction engine 230. The disclosed embodiments are not limited to any particular implementation of data storage 205. Consistent with disclosed embodiments, data storage 205 can be implemented using one or more relational databases, object-oriented or document-oriented databases, tabular data stores, graph databases, distributed file systems, or other suitable data storage options.

Consistent with disclosed embodiments, training engine 220 can be configured to train, or create and train, models. Training engine 220 can be configured to create models (e.g., in response to a command to create a trained model of a particular type using an input dataset) or obtain existing models from model storage 203. Training engine 220 can be configured to create or train models using training datasets obtained from data storage 205. In some embodiments, training engine 220 can be configured to store trained models in model storage 203.

Consistent with disclosed embodiments, training engine 220 can include model training and model evaluation components. Training engine 220 can be configured to train a model using a model training component and then determine performance measure values for the model using a model evaluation component.

In some embodiments, training engine 220 can provide a model and a cross-validation or holdout portion of a training dataset to the model evaluation component. In some embodiments, training engine 220 can specify one or more performance measures. Additionally, or alternatively, the model evaluation component can be configured with a predetermined or default set of performance measures. In some embodiments, the performance measures can include confusion matrices, mean-squared-error, mean-absolute-error, sensitivity or selectivity, receiver operating characteristic curves or area under such curves, precision and recall, F-measure, or any other suitable performance measure. In some embodiments, performance measure values can be displayed to a user through user device 299. The user may then interact through user device 299 with training engine 220 to update the model.

In some embodiments, training engine 220 can automatically update the model being trained based on the performance measure values. In various embodiments, training engine 220 can update the model being trained in response to user input provided through user device 299. Updating the model can include one or more of performing additional training (e.g., using the existing training dataset or another training dataset), modifying the model (e.g., changing the input features used by the model, changing the architecture of the model, or the like), or changing the training environment (e.g., changing training hyperparameters, changing a division of the training dataset into training, cross-validation, and holdout portions, or the like).

Consistent with disclosed embodiments, prediction engine 230 can be configured to predict the progression of cognitive impairment, or predict brain amyloid burden, for a subject using a prediction model. In some embodiments, prediction engine 230 can obtain the trained classification model from model storage 203. In some embodiments, prediction engine 230 can obtain input data for the subject from data storage 205. In some embodiments, prediction engine 230 can obtain the subject data from another data storage location. This alternative data storage location can be associated with another entity or user. For example, prediction engine 230 can receive or retrieve the subject data from a healthcare system controlled by an entity distinct from the entity that controls prediction engine 230.

Consistent with disclosed embodiments, the subject data can include baseline data. The baseline data can include demographic data, cognitive data, genomic data, imaging data, biomarker data, or other suitable baseline data. The prediction engine 230 can apply the baseline data to the trained prediction model to provide as output cognitive impairment progression data for the subject, or a prediction of brain amyloid burden. The output can be provided by prediction engine 230 to user device 299. In some embodiments, the output can be stored on a computing device associated with platform 200 or provided to another system.

Consistent with disclosed embodiments, user device 299 can provide a user interface for interacting with other components of platform 200. The user interface can be a graphical user interface. The user interface can enable a user to configure ETL engine 210 to extract, transform, and load data according to user specifications. The user interface can enable the user to specify how the transformed data received by dataset creation engine 215 is converted into labeled training data (or suitable patient data). In some embodiments, the user interface can enable the user to interact with dataset creation engine 215 to manually or semi-manually label or annotate the training data. In some embodiments, the user interface can enable a user to provide data or models to training engine 220 for training, or to prediction engine 230 for identification and classification.

In some embodiments, the user interface can enable a user to interact with training engine 220 to create or select a model for training, create or select a dataset for use in training the model, or select training parameters or hyperparameters. In some embodiments, the user interface can enable a user to interact with training engine 220 to display information related to training of the model (e.g., performance measure values, a change in loss function values during training, or other training information). In some embodiments, the user interface can enable a user to interact with prediction engine 230 to select a training model and patient data (e.g., a base image). In some embodiments, the user interface can enable a user to interact with prediction engine 230 to display any indication of an identified biological structure in the patient data, store the indication on a computing device, or transmit the indication to another system.

Components of platform 200 can be implemented using one or more computing devices. Such computing devices can include tablets, laptops, desktops, workstations, computing clusters, or cloud computing platforms. In some embodiments, components of platform 200 can be implemented using cloud computing platforms. For example, one or more of ETL engine 210, dataset creation engine 215, training engine 220, and prediction engine 230 can be implemented on a cloud computing platform. In some embodiments, components of platform 200 can be implemented using on-premises systems. For example, record(s) 201 or user device 299 can be, or be hosted on, on-premises systems. As an additional example, model storage 203 or data storage 205 can be, or be hosted on, on-premises systems.

Components of platform 200 can communicate using any suitable method. In some embodiments, two or more components of platform 200 can be implemented as microservices or web services. Such components can communicate using messages transmitted on a computer network. The messages can be implemented using SOAP, XML, HTTP, JSON, RCP, or any other suitable format. In some embodiments, two or more components of platform 200 can be implemented as software, hardware, or combined software/hardware modules. Such components can communicate using data or instructions written to or read from a memory (e.g., a shared memory), function calls, or any other suitable communication method.

As may be appreciated, the particular structure of platform 200 is not intended to be limiting. Consistent with disclosed embodiments, any two or more of record(s) 201, model storage 203, or data storage 205 can be combined, or hosted on the same computing device. Consistent with disclosed embodiments, ETL engine 210 and dataset creation engine 215 can be omitted from platform 200. In such embodiments, datasets formatted and configured for use by training engine 220 or prediction engine 230 can be deposited in data storage 205 by another system or using another method. Consistent with disclosed embodiments, ETL engine 210 and dataset creation engine 215 can be combined. In such embodiments, data extraction, transformation, and loading can be combined with feature extraction, labeling, and sample creation. Consistent with disclosed embodiments, training engine 220 and prediction engine 230 can be combined.

Though shown with one user device 299, platform 200 could have multiple user devices. Different user devices could be associated with different entities or different users having different roles. For example, user device 299 could be associated with a software engineer or data scientist who is developing the prediction model, while another user device could be associated with a clinician who is using the prediction model.

User device 299 can be combined with one or more other components of platform 200. In some embodiments, user device 299 and at least one of ETL engine 210, dataset creation engine 215, training engine 220, or prediction engine 230 can be implemented by the same computing device. In various embodiments, user device 299 and at least one of model storage 203 or data storage 205 can be implemented by the same computing device.

As may be appreciated, platform 200 can be integrated into a method for treating subjects or for conducting clinical trials. Prediction engine 230 can use a trained prediction model and input data for the subject to predict brain amyloid burden or cognitive impairment progression for the subject. The predicted brain amyloid burden or cognitive impairment progression can be used to determine a patient treatment plan for the patient. In some embodiments, the cognitive impairment progression for the subject can be used as a prognostic covariate in determining a treatment effect in a clinical trial.

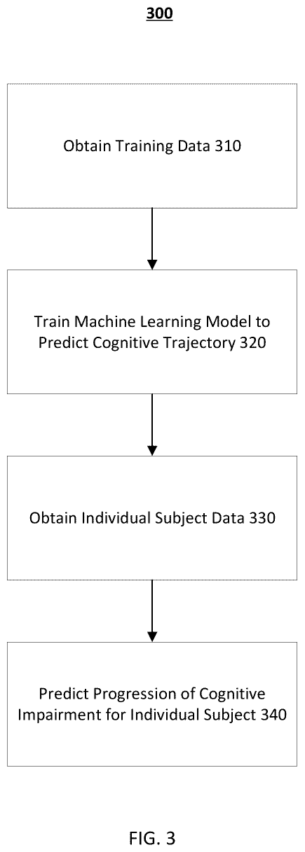

FIG. 3 depicts an exemplary process 300 of predicting the progression of cognitive decline in a subject, consistent with disclosed embodiments. For convenience of description, process 300 is described as being performed using platform 200. However, process 300 can also be performed at least in part using another computing system. Process 300 can include a dataset creation phase, a training phase, and a prediction phase. In the dataset creation phase, components of platform 200 (e.g., ETL engine 210, dataset creation engine 215, or the like) can obtain training data from databases (e.g., record(s) 201, or the like). In the training phase, components of platform 200 (e.g., training engine 220, or the like) can create or refine predictive models for predicting cognitive impairment progression data from baseline data. In the prediction phase, components of platform 200 (e.g., prediction engine 230, or the like) can use prediction model(s) generated in the training phase to predict cognitive impairment progression data from baseline data for a subject. The predicted cognitive impairment progression data can be used manage treatment for the subject, or can be used as a prognostic covariate in a clinical trial.

In step 310 of process 300, components of platform 200 (e.g., ETL engine 210, dataset creation engine 215, or the like) can obtain training data, consistent with disclosed embodiments. The training data can concern subjects satisfying a cognitive impairment condition. The cognitive impairment condition can specify that the subjects have a diagnosis of a neurological disease, dysfunction, or injury (e.g., a diagnosis of AD, a diagnosis of MCI, a diagnosis of dementia, or the like), have certain signs (e.g., amyloid positivity on an PET scan; a biomarker score, such as a plasma, serum, or cerebrospinal fluid p-Tau181, Aβ1-42 score, or Aβ1-40 score; or the like). In some instances, the cognitive impairment condition can be an inclusion criterion of a clinical study.

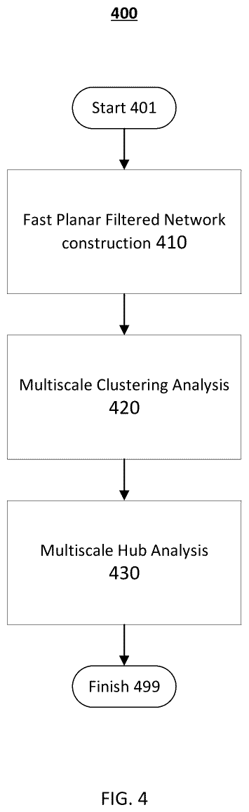

In some embodiments, the components of platform 200 can obtain at least a portion of the training data from a database (e.g., record(s) 201 or the like) or another system. In some embodiments, the components of platform 200 can generate at least a portion of the training data. For example, dataset creation engine 215 can identify brain regions as hubs and clusters of brain regions as modules. Dataset creation engine 215 can identify such brain regions using network analysis or multi-level clustering. For example, as described herein with regards to FIG. 4, dataset creation engine 215 can identify such regions using MEGENA. Dataset creation engine 215 can generate composite values for the modules based on the measurements for the brain regions comprising the modules.

In some embodiments, the training data can include baseline data for the subjects, consistent with disclosed embodiments. In some embodiments, the baseline data can include cognitive data and image data, as described herein. For example, the image data can include measurements for one or more brain regions identified as hubs and composite values for one or more clusters of brain regions identified as modules. In some embodiments, the baseline data can include demographic data, as described herein. In some embodiments, the baseline data can include genomic data, as described herein. For example, the baseline data can include ApoE4 allelic count. In some embodiments, the baseline data can include biomarker data. In some embodiments, the biomarker data can be plasma, serum, or cerebrospinal fluid biomarker data.

In some embodiments, the training data can include cognitive impairment progression data for the subjects, consistent with disclosed embodiments. The cognitive impairment progression data can include repeated measurements acquired over time. In some embodiments, the repeated measurements can be cognitive measurements, as described herein, or can depend on such cognitive measurements. In some embodiments, the repeated measurements can be expressed as a function of a baseline cognitive measurement and a subsequent cognitive measurement (e.g., a difference between a baseline cognitive assessment and a subsequent cognitive assessment). For example, a repeated measure can be or include a change in CDR-SB measurement, ADCOMS measurement, ADAS measurement, or the like for a subject. In some embodiments, each repeated measurement can include, or depend upon, a clinical dementia sum of boxes (CDR-SB) measurement, an Alzheimer's Disease Composite Score (ADCOMS) measurement; or an Alzheimer's Disease Assessment Scale (ADAS) measurement.

As may be appreciated, the repeated measurements for a subject can be acquired after the acquisition of the baseline cognitive data for the subject. In some embodiments, the repeated measurements can be acquired at assessments repeatedly conducted after the baseline cognitive data is acquired. Such assessments may be performed, and repeated measurements acquired, at time intervals of between 3 and 12 months for each subject. In some embodiments, the elapsed time between acquisition of the baseline cognitive data for the first subject and acquisition of a final one of the repeated measurements can be between 12 and 36 months, or between 18 months and 24 months, or the like.

In some embodiments, the elapsed time associated with a repeated measure can be implicit. For example, the repeated measurements can be expressed as a vector (or matrix in case of vector-valued repeated measurements), with the elapsed time implicit in the position of the repeated measurement in the vector (or the column of the repeated measurement in the matrix). In some embodiments, the elapsed time associated with a repeated measure can be implicit. For example, the repeated measurements can be expressed as tuples, with each tuple including the repeated measurement(s) and an indication of the elapsed time since the baseline assessment.

In step 320 of process 300, components of platform 200 (e.g., training engine 220, or the like) can train a predictive model to predict the progression of cognitive impairment of a subject, consistent with disclosed embodiments. In some embodiments, training engine 220 can create the predictive model and then store the predictive model in model storage 203. In some embodiments, training engine 220 can obtain a predictive model from model storage 203, or another database or system, and then refine the model.

In some embodiments, training engine 220 can obtain hyperparameters for training the predictive model. The particular hyperparameters obtained can depend on the type of predictive model and the disclosed embodiments are not limited to any particular set of hyperparameters. For example, a neural network model may have hyperparameters governing layer arrangement and configuration, batch size, dropout, or the like. As an additional example, a gradient boosted model may have hyperparameters governing learning rate, number of trees, bagging fraction, tree depth, or the like.

In some embodiments, a user can interact with user device 299 to provide hyperparameters to training engine 220. In some embodiments, training engine 220 can receive or retrieve hyperparameters from another component of platform 200. In some embodiments, training engine 220 can generate suitable hyperparameters. For example, training engine 220 can be configured to conduct an iterative or adaptive search of a predetermined hyperparameter space (e.g., through training predictive models, evaluating the performance of the models, and updating the selected hyperparameters based on the performance of the models).

In some embodiments, training engine 220 can train the predictive model using the hyperparameters and the training data obtained in step 310. The disclosed embodiments are not limited to any particular code or instructions for training the model. For example, when training engine 220 uses the R statistical package and the predictive model is a gradient boosted model, the following code can be used to train the model:

```
  library(gbm)
  x = TrainingData[,xvar]
  y = TrainingData [,yvar]
  train.data = cbind(y, x)
  set.seed(263)
  gbm.fit <− gbm(y ~ ., data=train.data,
verbose = FALSE, distribution = “gaussian”,
shrinkage = 0.01,
interaction.depth = 3,
n.minobsinnode = 100,
n.trees = 1000,
cv.folds = 5,
bag.fraction = 0.5,
n.cores=1
)
```

Training engine 220 can execute this code to determine a predictive gradient boosted model using the training data and the given hyperparameter values.

In some embodiments, training engine 220 can be configured to evaluate the performance of multiple model designs using the same training dataset. The performance of a model design can be determined using k-fold cross validation. In some embodiments, training engine 220 can be configured to evaluate the performance of the best-performing model design by dividing the training dataset into training and validation subsets. Training engine 220 can evaluate model designs by performing k-fold cross validation using the training subset. Training engine 220 can select a model design and evaluate the performance of that model design using the validation subset.

In step 330 of process 300, components of platform 200 (e.g., ETL engine 210, dataset creation engine 215, prediction engine 230, or the like) can obtain baseline data for an individual subject. In some embodiments, the individual subject can satisfy a cognitive impairment condition. For example, the individual subject and the subjects from which the training data was obtained can satisfy the same cognitive impairment condition. In some embodiments, the individual subject can satisfy a similar or equivalent cognitive impairment condition (e.g., the training subject may have had a diagnosis of AD, while the individual subject may have clinical findings suggestive of AD). In some embodiments, the components of platform 200 can obtain at least a portion of the individual subject data from a database (e.g., record(s) 201 or the like) or from another system. For example, platform 200 can be configured to accept prediction requests from other systems. In some embodiments, the components of platform 200 can generate at least a portion of the individual subject data.

In some embodiments, the baseline data for the individual subject (e.g., the prediction baseline data) can be the same as the baseline data included in the training data (e.g., the training baseline data). For example, when the training baseline data includes a combination of a certain demographic data, biomarker data, and cognitive measurements, the prediction baseline data can include the same demographic data, biomarker data, and cognitive measurements. As may be appreciated, obtaining the prediction baseline data can include reformatting or arranging the prediction baseline data to match the format or arrangement of the training baseline data. Similarly, obtaining the prediction baseline data can include handling missing values or erroneous values in the training baseline data. Furthermore, when obtaining the training baseline data includes generating certain values (e.g., generating composite values for modules), obtaining the prediction baseline data can similarly include generating these values.

In some embodiments, the prediction data can include an elapsed time. In some embodiments, a user can interact with platform 200 to request a prediction of cognitive impairment at this elapsed time.

In step 340 of process 300, components of platform 200 (e.g., prediction engine 230, or the like) can predict the progression of cognitive impairment for the subject, consistent with disclosed embodiments. In some embodiments, prediction engine 230 can input the prediction baseline data to the trained prediction model. The output of the trained prediction model can be a sequence of predicted cognitive measurements (e.g., predicted cognitive impairment progression data). The predicted cognitive impairment progression data can be implicitly or expressly associated with elapsed times since the baseline. For example, the output can be a vector (or matrix) of values, with each position in the vector (or column of the matrix) being implicitly associated with an elapsed time. As an additional example, the predicted cognitive impairment progression data can be a set of tuples, each tuple including an elapsed time and a set of predicted cognitive measurements for that elapsed time.

Consistent with disclosed embodiments, platform 200 can provide the predicted cognitive impairment progression data. Platform 200 can provide the predicted cognitive impairment progression data to a user of platform 200 (e.g., by providing the predicted cognitive measurements to user device 299 for display), store the predicted cognitive impairment progression data in a component of platform 200, provide the predicted cognitive impairment progression data to another system (e.g., a system that provided a prediction request), or the like.

As may be appreciated, the predicted cognitive impairment progression data may be manually, semi-automatically, or automatically assessed for an indication of progression of neurological disease, dysfunction, or injury. In some instances, for example, the subject may have been diagnosed with mild cognitive impairment. Satisfaction of the cognitive impairment condition by the subject may have depended on such a diagnosis (or, for example, clinically equivalent findings). The predicted cognitive impairment progression data may provide an indication that the subject will progress to Alzheimer's disease. Platform 200 may be configured to automatically evaluate (e.g., using baseline data, predicted cognitive measurements, and diagnostic thresholds, or the like) the predicted cognitive impairment progression data and provide an indication of such a predicted progression.

As may be appreciated, a trained predictive model can be used to screen or select patients for inclusion in a clinical trial. Cognitive impairment progression data can be predicted for a candidate patient using baseline data acquired for that candidate patient. The candidate patient can be included in the study when the predicted cognitive impairment progression data satisfies a selection criterion. In various embodiments, the selection criterion can depend on a final cognitive measurement, a change in cognitive measurements between a baseline cognitive measurement and the final cognitive measurement, a cognitive measurement at specified time after baseline, values or coefficients of a function fit to the predicted cognitive impairment progression data, or another suitable measure. For example, a patient may be included in a clinical trial when a final predicted cognitive measurement in the predicted cognitive impairment progression data exceeds a threshold value or is within a specified range. Such patients may have a greater need for treatment (as they otherwise would likely experience greater cognitive decline).

As may be appreciated, a trained predictive model can be used in study design. As described herein, a clinical trial population can be enriched with patients likely to benefit from a treatment. In particular, a trained predictive model can be used to screen or select patients for inclusion in a clinical trial. The patients selected can be those likely to exhibit at least a threshold amount of cognitive decline. As may be appreciated, treatment effect, study size, and study power can be related. By selecting patients likely to exhibit substantial cognitive decline, fewer patients can be enrolled, or study power can be increased, or detectable treatment effect size reduced, or some combination of the foregoing.

Furthermore, the benefits of treatment for such patients may be more readily apparent than for less-afflicted patients. Because the effects of treatment are more apparent (e.g., treatment effects are larger), screening or selecting patients using a trained predictive model can enable an improved clinical trial design: the number of patients enrolled can be reduced, the minimum detectable treatment effect can be increased, study power can be increased, or some combination of the foregoing.

As may be appreciated, the predicted cognitive impairment progression data can be used to evaluate the effect of a treatment for neurological disease, dysfunction, or injury in a clinical study. The clinical study may include multiple participants. The participants can be screened for satisfaction of a cognitive impairment condition and baseline data can be acquired for each participant. The participants can be assigned to either a control or a treatment group of the study.

Using the trained predictive model, cognitive impairment progression data can be predicted for one or more participants in the treatment group of the study and used as a prognostic covariate in analyzing the results of the study.

For example, the clinical trial can concern an Alzheimer's treatment. The trained predictive model can be used to predict cognitive impairment progression data for at least some patients assigned to the treatment group of the clinical trial. The predicted cognitive impairment progression data can be used as a prognostic covariate in determining an effect of the Alzheimer's treatment.

FIG. 4 depicts a process 400 for identifying hubs and modules in the brain based on brain region measurements, consistent with disclosed embodiments. The brain region measurements can include volume, area, cortical thickness, or the like. Process 400 can be used as part of process 300, describe herein, or as part of another process. As described herein, training baseline data can include image data, such as brain region measurements associated with brain regions of subjects. Process 400 can be used to identify particular brain regions (e.g., hubs) or particular combinations of brain regions (e.g., modules) that are particularly representative of the brain region measurements for a subject. In this manner, process 400 can reduce the dimensionality of the training baseline data and improve the robustness of the trained predictive model.

Process 400 is described herein as being performed by dataset creation engine 215 of platform 200 for convenience of disclosure. However, this description is not intended to be limiting. Without departing from envisioned embodiments, process 400 can be performed by another component of platform 200, or by another system. Likewise, this process is described as being performed using MRI brain region measurements. However, brain region measurements obtained using any suitable imaging modality can be used, without departing from envisioned embodiments.

In step 401, process 400 can start. Dataset creation engine 215 can obtain MRI regional measures for training subjects. Dataset creation engine 215 can receive or retrieve the MRI regional measures from another component of platform 200 (e.g., data storage 205, or the like), or another system. Dataset creation engine 215 can generate the MRI regional measures from MRI image data for the training subjects (e.g., using FREESURFER, or another suitable tool for the analysis and visualization of neuroimaging data), which dataset creation engine 215 can in turn receive or retrieve from another component of platform 200 (e.g., data storage 205, or the like), or another system. The MRI regional measures can correspond to regions specified in a neuroanatomical atlas (e.g., the Desikan-Killiany atlas, Harvard-Oxford atlas, Automated Anatomical Labeling atlas, Brainnetome atlas, or the like). In some embodiments, dataset creation engine 215 can normalize volume and area measures by intra-cranial volume to reduce inter-subject variability and account for variance due to head size.

In step 410 of process 400, dataset creation engine 215 can construct a planar-filtered network graph using the MRI regional measures. The planar-filtered network graph can include nodes corresponding to brain regions and edges corresponding to relationships between the brain regions. For example, an edge between two nodes can correspond to correlations between brain region measurements for the regions for the training subjects. The dataset creation engine 215 can determine a correlation of MRI measures across all pairs of brain regions. The pairs of regions can be ranked by correlation and then filtered based on a false discovery rate threshold. The filtered pairs can be iteratively tested for planarity (e.g., using the Boyer-Myrvold algorithm, or the like). If a pair passes the planarity test, then the network can be updated to include a link corresponding to the pair in the network. The embedding process can be repeated until a termination condition is satisfied. The termination condition can depend on the number of pairs included in the network (e.g., whether the number of pairs included is the maximal number of edges that can be embedded on a topological sphere, such that every edge can be drawn without crossing another), whether any pairs remain untested, or the number of pairs rejected for each pair accepted. In this manner, the dataset creation engine 215 can generate a planar-filtered network graph that favors inclusion of more highly correlated brain regions.

In step 420 of process 400, dataset creation engine 215 can perform a multi-level clustering analysis using the planar-filtered network graph. The clustering analysis can attempt to optimize within-cluster compactness, local clustering structures, and overall modularity. The clustering analysis can be performed iteratively. In an initial iteration, the clustering analysis can include dividing the network graph into clusters of nodes. In each subsequent iteration, the embedded network can perform a nested split on each cluster of nodes.

In some embodiments, the nested split can be performed using k-medoids clustering (or another suitable clustering into a predetermined number of clusters) according to shortest path distances (and optionally with cluster boundaries refined using local path indices), with k selected through an iterative process. In each iteration, the value of k can be different, and the resulting clustering can be evaluated using a measure of clusteredness on networks (e.g., Newman's modularity, or the like). Different values of k can be attempted until a threshold condition is satisfied. The threshold condition can depend on the number of k values investigated, on the number of k values investigated since the last k value that resulted in a best-achieved compactness measure, a timing or duration condition, or another suitable condition. A candidate split for a cluster can be the split having the best-achieved compactness measure.

In some embodiments, the candidate split for a cluster can be accepted or rejected based on a compactness measure determined for each sub-cluster within the split. The compactness measure for a sub-cluster can depend on the path distances within the sub-cluster (e.g., a normalized average shortest path distance, or the like) and a scaling parameter. In some embodiments, the candidate split for a cluster can be rejected when the compactness measure for all sub-clusters within the cluster are greater than the compactness measure for the cluster. Otherwise, the candidate split can be accepted.

In some embodiments, a statistical significance can be associated with sub-clusters. This statistical significance can depend on the value of the scaling parameter necessary to accept the sub-cluster. The statistical significance can be the likelihood of randomly generating a sub-cluster having at least the value of the compactness measure for the sub-cluster given the value of the scaling parameter necessary to accept the sub-cluster.

In some embodiments, nested splits can be performed until a terminal condition is satisfied. In some embodiments, the termination condition can be satisfied when no sub-clusters of a parent cluster can be identified that are more compact than the parent cluster for any value of the scaling parameter. In some embodiments, the termination condition can be satisfied when no sub-clusters demonstrate statistical significance greater than a threshold value (e.g., 0.05, or another suitable significance value). As may be appreciated, the disclosed embodiments are not limited to these particular termination conditions. Other suitable termination conditions (e.g., time or resource-based termination conditions) can also be used. In some embodiments, the identified clusters can be the modules.

In step 430 of process 400, dataset creation engine 215 can perform a multiscale hub analysis of the embedded network to identify related brain regions at each scale defined by the above scaling parameter and across all scales. In a first step, values of the scaling parameter associated with significant clusters (from step 420) can be in turn clustered (e.g., using k-medoids clustering or another clustering method), based on within-cluster node connectivity at the different scaling parameters. In a second step, a significance of a node can be identified for each scale using the within-cluster connectivity of the node at that scale and within-cluster connectivities of nodes in randomly generated sub-networks for that scale. In a third step, hubs can be identified by combining significance scores of individual brain regions across all different scales.

In step 499, process 400 can terminate. Dataset creation engine 215 can generate composite values for the identified modules using the MRI regional measures for these modules. In some embodiments, a first principal component can be calculated for a measurement type (e.g., volume, surface area, cortical thickness, or the like) over all brain regions included in the module. This first principal component can then be associated with the module. As may be appreciated, a principal component value can be generated for multiple types of measurements (e.g., each of volume, surface area, cortical thickness, or the like) and the results for these types of measurements can be associated with the model. As may be appreciated, the disclosed embodiments are not limited to using principal component analysis to generate values for modules.

When process 400 is performed as part of process 300, the regional values for the identified hubs and modules can be used as input data for training the prediction models, consistent with disclosed embodiments.

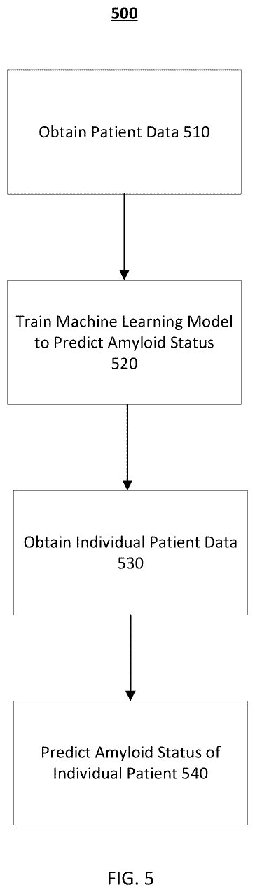

FIG. 5 depicts a process 500 for predicting brain amyloid status of a subject, consistent with disclosed embodiments. For convenience of description, process 500 is described as being performed using platform 200. However, process 500 can also be performed at least in part using another computing system. Process 500 can include a dataset creation phase, a training phase, and a prediction phase. In the dataset creation phase, components of platform 200 (e.g., ETL engine 210, dataset creation engine 215, or the like) can obtain training data from databases (e.g., record(s) 201, or the like). In the training phase, components of platform 200 (e.g., training engine 220, or the like) can create or refine predictive models for predicting brain amyloid status from baseline data. In the prediction phase, components of platform 200 (e.g., prediction engine 230, or the like) can use prediction model(s) generated in the training phase to predict brain amyloid status data from baseline data for a subject. The predicted brain amyloid status data can be used manage treatment for the subject, or can be used as a prognostic covariate in a clinical trial.

In step 510 of process 500, components of platform 200 (e.g., ETL engine 210, dataset creation engine 215, or the like) can obtain training data, consistent with disclosed embodiments. The training data can concern subjects satisfying a cognitive impairment condition. The cognitive impairment condition can specify that the subjects have a diagnosis of a neurological disease, dysfunction, or injury (e.g., a diagnosis of AD, a diagnosis of MCI, a diagnosis of dementia, or the like), have certain signs (e.g., amyloid positivity on an PET scan; a biomarker score, such as a plasma, serum, or cerebrospinal fluid p-Tau181, Aβ1-42 score, or Aβ1-40 score; or the like). In some instances, the cognitive impairment condition can be an inclusion criterion of a clinical study.

In some embodiments, the components of platform 200 can obtain at least a portion of the training data from a database (e.g., record(s) 201 or the like) or another system. In some embodiments, the components of platform 200 can generate at least a portion of the training data.

In some embodiments, the training data can include baseline data for the subjects, consistent with disclosed embodiments. The baseline data can include cognitive data and image data, as described herein. In some embodiments, the baseline data can include demographic data, as described herein. In some embodiments, the baseline data can include genomic data, as described herein. For example, the baseline data can include ApoE4 allelic count. In some embodiments, the baseline data can include biomarker data. In some embodiments, the biomarker data can be plasma, serum, or cerebrospinal fluid biomarker data.

In some embodiments, the training data can include brain amyloid data for the subjects, consistent with disclosed embodiments. The brain amyloid data can include an assessment of brain amyloid burden based on image data for the subjects. As described herein, the image data can be or include PET image data showing tracer uptake, or image data acquired using another suitable image modality. In some embodiments, the brain amyloid data can be classification data (e.g., a binary classification representing the satisfaction of a diagnostic criterion, a multi-class classification indicating stages or classes of amyloid plaque deposition, or the like). In some embodiments, the brain amyloid data can be continuously valued data, such as intensity data, detected amount data (e.g., number of pixels satisfying a detection criterion), or other continuously valued data extracted from the image data for the subjects.

In step 520 of process 500, components of platform 200 (e.g., training engine 220, or the like) can train a predictive model to predict brain amyloid status for a subject, consistent with disclosed embodiments. In some embodiments, training engine 220 can create the predictive model and then store the predictive model in model storage 203. In some embodiments, training engine 220 can obtain a predictive model from model storage 203, or another database or system, and then refine the model.

In some embodiments, training engine 220 can obtain hyperparameters for training the predictive model. The particular hyperparameters obtained can depend on the type of predictive model and the disclosed embodiments are not limited to any particular set of hyperparameters. For example, a neural network model may have hyperparameters governing layer arrangement and configuration, batch size, dropout, or the like. As an additional example, a gradient boosted model may have hyperparameters governing learning rate, number of trees, bagging fraction, tree depth, or the like.

In some embodiments, a user can interact with user device 299 to provide hyperparameters to training engine 220. In some embodiments, training engine 220 can receive or retrieve hyperparameters from another component of platform 200. In some embodiments, training engine 220 can generate suitable hyperparameters. For example, training engine 220 can be configured to conduct an iterative or adaptive search of a predetermined hyperparameter space (e.g., through training predictive models, evaluating the performance of the models, and updating the selected hyperparameters based on the performance of the models).

In some embodiments, training engine 220 can train the predictive model using the hyperparameters and the training data obtained in step 510. The disclosed embodiments are not limited to any particular code or instructions for training the model. In some embodiments, training engine 220 can be configured to evaluate the performance of multiple model designs using the same training dataset (e.g., Monte-Carlo Logistic Lasso models, Bayesian Logistic Elastic Net models, regularized random forest models, stochastic gradient boosting machine models, or the like). The performance of a model design can be determined using k-fold cross validation. In some embodiments, training engine 220 can be configured to evaluate the performance of the best-performing model design by dividing the training dataset into training and validation subsets. Training engine 220 can evaluate model designs by performing k-fold cross validation using the training subset. Training engine 220 can select a model design and evaluate the performance of that model design using the validation subset.

In step 530 of process 500, components of platform 200 (e.g., ETL engine 210, dataset creation engine 215, prediction engine 230, or the like) can obtain baseline data for an individual subject. In some embodiments, the components of platform 200 can obtain at least a portion of the individual subject data from a database (e.g., record(s) 201 or the like) or from another system. For example, platform 200 can be configured to accept prediction requests from other systems. In some embodiments, the components of platform 200 can generate at least a portion of the individual subject data.

In some embodiments, the baseline data for the individual subject (e.g., the prediction baseline data) can be the same as the baseline data included in the training data (e.g., the training baseline data). For example, when the training baseline data includes a combination of a certain demographic data, biomarker data, and cognitive measurements, the prediction baseline data can include the same demographic data, biomarker data, and cognitive measurements. As may be appreciated, obtaining the prediction baseline data can include reformatting or arranging the prediction baseline data to match the format or arrangement of the training baseline data. Similarly, obtaining the prediction baseline data can include handling missing values or erroneous values in the training baseline data. Furthermore, when obtaining the training baseline data includes generating certain values (e.g., generating composite values for modules), obtaining the prediction baseline data can similarly include generating these values.

In step 540 of process 500, components of platform 200 (e.g., prediction engine 230, or the like) can predict brain amyloid status for a subject, consistent with disclosed embodiments. In some embodiments, prediction engine 230 can input the prediction baseline data to the trained prediction model. The output of the trained prediction model can be a prediction of brain amyloid status for the subject. As may be appreciated, the type of prediction can depend on how the model is trained. When the training baseline data include class-valued brain amyloid data, the output of the prediction model can be predicted classes (or class likelihoods). When the training baseline data include continuously valued brain amyloid data, the output of the prediction model can be a predicted brain amyloid data value.

Consistent with disclosed embodiments, platform 200 can provide the predicted cognitive measurements. Platform 200 can provide the predicted cognitive measurements to a user of platform 200 (e.g., by providing the predicted output class, class probabilities, or brain amyloid data values to user device 299 for display), or store the predicted cognitive measurements in a component of platform 200, provide the predicted cognitive measurements to another system (e.g., a system that provided a prediction request), or the like.

### EXAMPLES

Multiple investigations were performed into the training and use of predictive models consistent with disclosed embodiments. These investigations concerned both prediction of a progression of cognitive impairment and prediction of brain amyloid status.

### Example 1

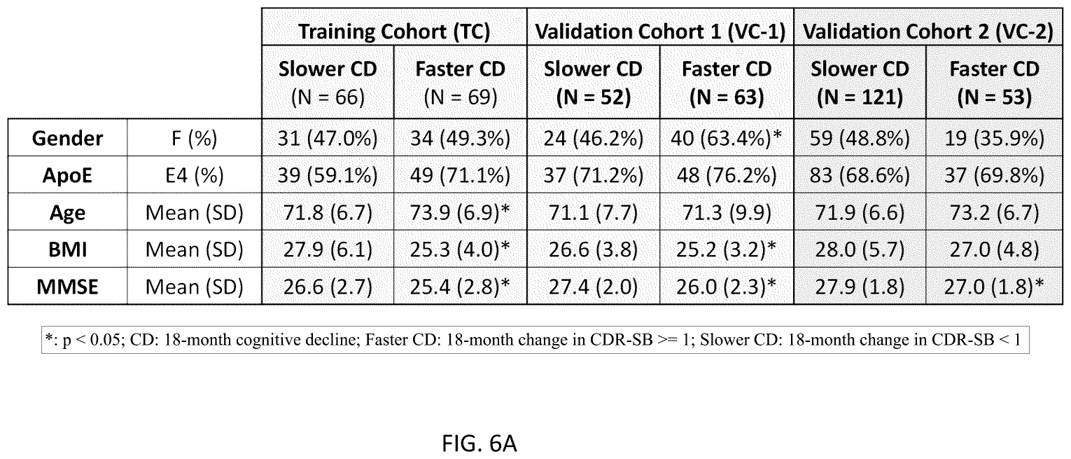

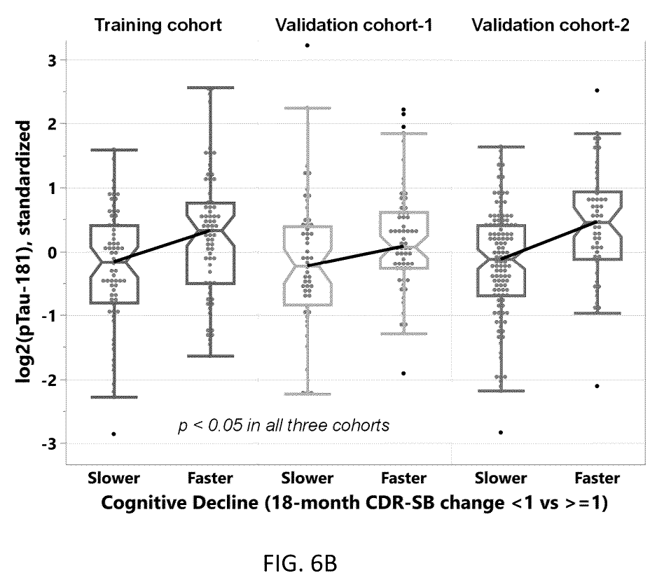

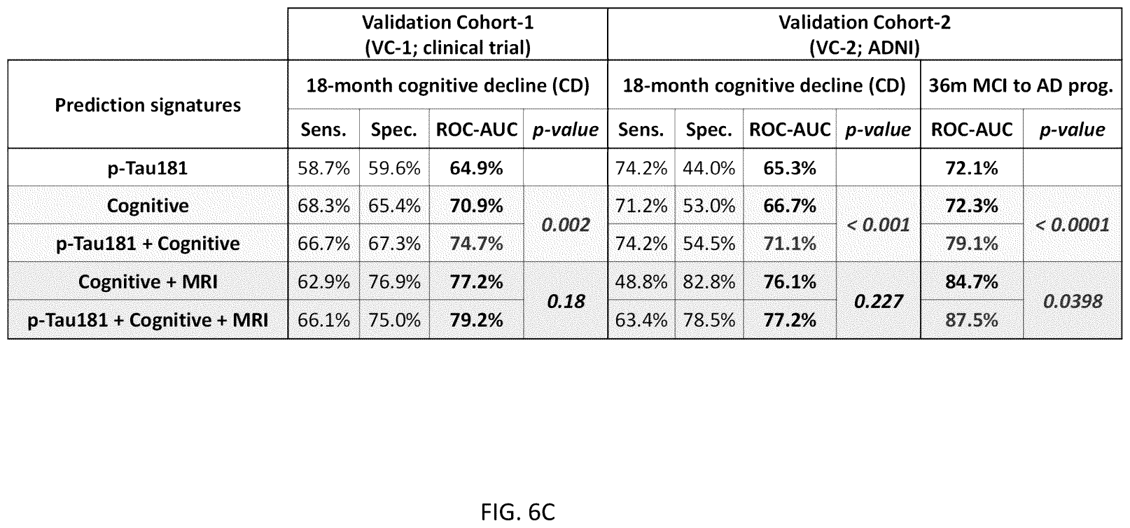

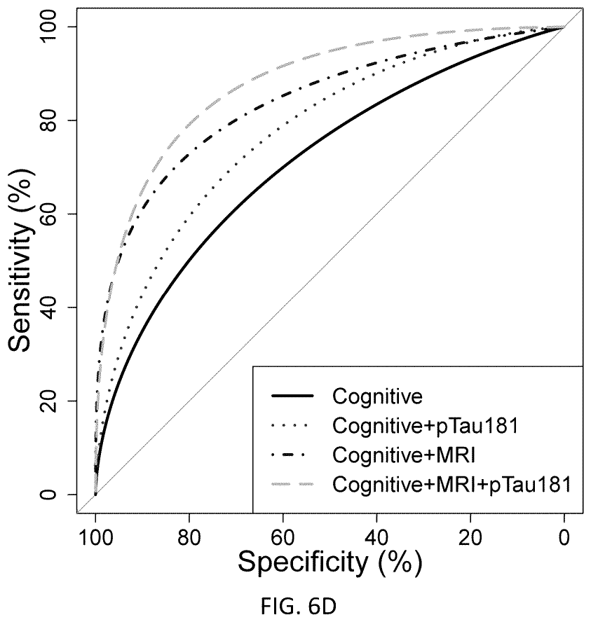

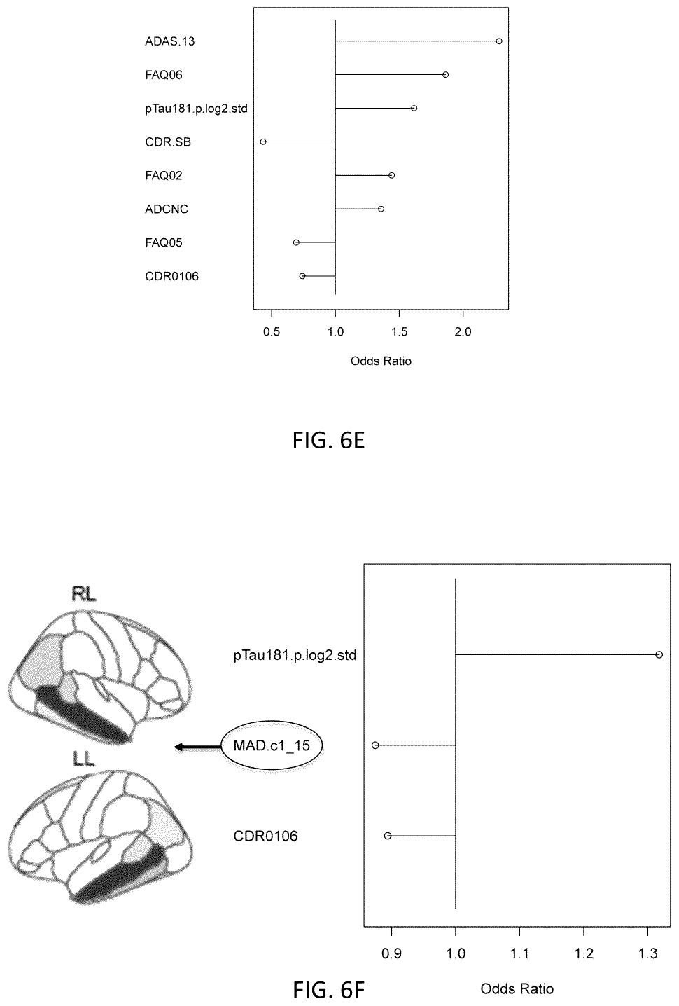

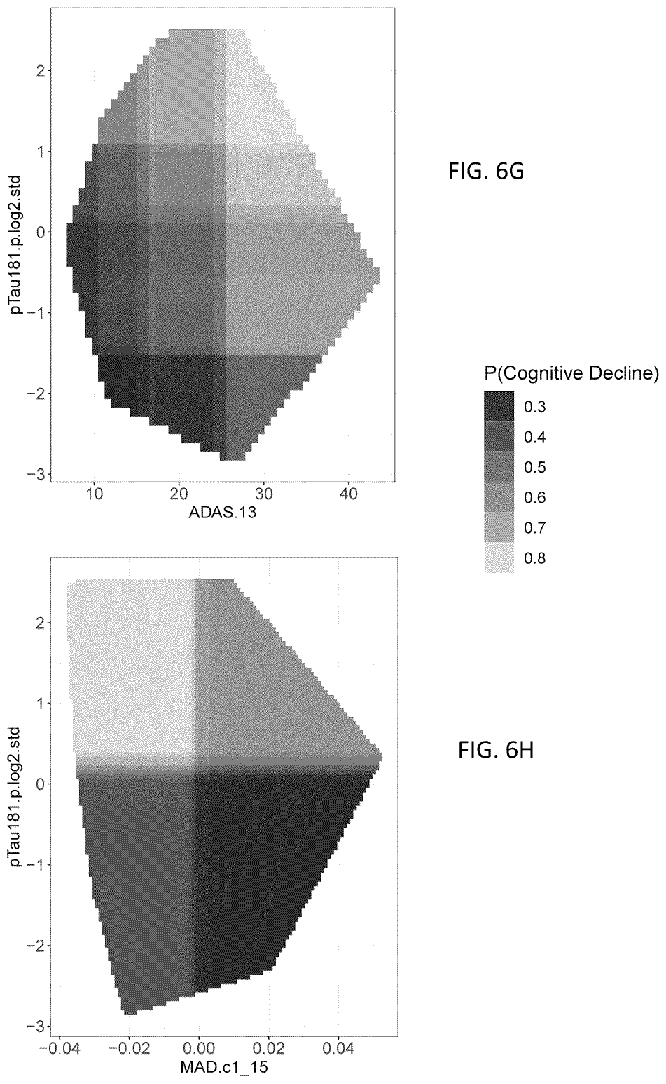

FIGS. 6A to 6H concern an investigation into the prediction of cognitive impairment progression and disease progression in amyloid positive subjects with mild cognitive impairment (e.g., A+ MCI subjects). In particular, the investigation considered whether plasma p-Tau181, a biomarker indicative of brain amyloid burden, in combination with demographic factors and other inputs could predict disease progression in A+ MCI subjects. As may be appreciated, blood-based tests for screening and monitoring subjects in Alzheimer's disease (AD) clinical trials would be faster, easier, and more cost-effective, compared to conventional CSF and imaging methods.

FIG. 6A depicts a demographic summary of training and validation cohorts used in the creation of a model of patient neurological disease progression. The training cohort used to construct the model included 135 A+ MCI placebo subjects from two clinical trials. Two independent validation cohorts for testing the performance of these signatures included 115 and 174 A+ MCI subjects respectively from the placebo arm of another clinical trial (VC-1) and from the Alzheimer's Disease Neuroimaging Initiative (ADNI) database (VC-2). Subjects with slower cognitive impairment progression that dropped out before month-18 were excluded.

Patient data included biomarker data. Plasma p-Tau181 was measured using Simoa assay at three different sites. These measurements were normalized to have similar means and variances to make them comparable across cohorts (i.e., by subtracting means and dividing by standard deviation). The training cohort and the first validation cohort included 18-month clinical follow-up, while the second validation cohort included 3 to 10-year follow-up. The threshold for faster cognitive impairment progression was set at an 18-month change in clinical dementia rating sum of boxes (CDR-SB) greater than or equal to 1.

Baseline MMSE was significantly lower in subjects with faster cognitive impairment progression in all cohorts (p<0.05). Baseline BMI was significantly lower in subjects that experienced faster cognitive impairment progression in the training cohort and in one of the validation cohorts (p<0.05). Subjects in the training cohort with faster cognitive impairment progression were significantly older.

In this investigation, multiple predictive models were constructed for predicting cognitive impairment progression. The predictive models were Bayesian Elastic-Net (BEN), regularized random forests, and gradient boosting models.

Additional predictive models were derived for assessing the added value of ApoE4 status, cognitive function assessments, and brain region measurements from magnetic resonance imaging (MRI). Demographics (age, sex, and BMI) were considered in all evaluated models. Performance of these models was first assessed via 10 iterations of 10-fold stratified cross-validation within the training cohort, and then tested in the first and second validation cohorts.

Among the machine-learning algorithms considered, BEN performed the best for predicting 18-month Alzheimer's disease progression in the two independent validation cohorts. For predicting 18-month cognitive impairment progression, baseline plasma p-Tau181 achieved similar performance as baseline cognitive function, with area under the receiver operating characteristic curve (ROC-AUC) of 64.9% and 70.9%, respectively, in VC-1 (p=0.199) and 65.3% and 66.7%, respectively, in VC-2 (p=0.395).

Overall, predictive models consistent with disclosed embodiments identified A+ MCI subjects likely to experience faster cognitive impairment progression over an 18-month interval. These predictive models used baseline plasma p-Tau181 levels as an input and demonstrated improved performance when baseline plasma p-Tau181 levels were combined with baseline cognitive function measures. These predictive models also demonstrated improved performance in predicting 36-month progression of MCI subjects to AD when using baseline plasma p-Tau181 levels in combination with baseline cognitive function or brain region MRI features.

FIG. 6B depicts a comparison of biomarker levels in subjects experiencing slower and faster cognitive impairment progression. Baseline p-Tau181 was significantly elevated in subjects with faster cognitive decline (CD) in all three cohorts (training, first validation, and second validation).

FIG. 6C depicts a performance summary of predictive models derived from a BEN model for predicting 18-month CD and 36-month progression to AD in VC-1 and VC-2. Demographics (age, sex, BMI) were considered in all predictive models. In addition to demographics, the models used as inputs baseline p-Tau181 (row 1), cognitive function (row 2), the combination of baseline p-Tau181 and cognitive function (row 3), the combination of cognitive function and MRI imaging data (row 4), and the combination of cognitive function, MRI imaging data, and baseline p-Tau181 (row 5). Adding ApoE4 did not improve prediction performance.

FIG. 6D depicts ROC curves for predicting 36-month MCI to AD progression in the second validation cohort. As depicted in FIG. 6D and shown in FIG. 6C, combining baseline plasma p-Tau181 with cognitive function or brain region MRI significantly improved ROC-AUC.

FIG. 6E depicts the relative importance (e.g., odds ratio) of certain significant inputs to a BEN model for predicting CD at 18 months based on biomarker and cognitive function inputs. The significant inputs in this model included the 13-item ADAS-Cog composite score (ADAS-13), the Functional Activities Questionnaire Scores (subparts 2, 5, 6), a function of the patient plasma p-Tau181 level (e.g., a standardized, log 2-transformed plasma p-Tau181 level), the ADCNC-number cancellation subscore, and the CDR0106-personal care subscore.

FIG. 6F depicts the relative importance (e.g., the odds ratio) of certain significant inputs to a BEN model for predicting CD at 18 months based on biomarker, cognitive function, and brain imaging data inputs. The significant inputs in this model included a function of the patient plasma p-Tau181 level (e.g., a standardized, log 2-transformed plasma p-Tau181 level), the CDR0106-personal care subscore, and a combination of MRI features automatically selected by the BEN model as being most useful for predicting cognitive impairment progression. These brain regions were related to inferior parietal, inferior temporal, middle temporal, and banks of the superior temporal sulcus regions. These brain regions are depicted in a brain heatmap in FIG. 6F. The right lateral (RL) and left lateral (LL) images are the right and left lateral views.

FIGS. 6G and 6H depict heatmaps showing interactions between certain input values and a predicted likelihood of cognitive impairment progression for a gradient boosting machine algorithm. FIG. 6G depicts the interaction between patient plasma p-Tau181 level (e.g., a standardized, log 2-transformed plasma p-Tau181 level) and cognitive level (e.g., ADAS-13). FIG. 6H depicts the interaction between patient plasma p-Tau181 level and a combination of brain region MRI features related to inferior parietal, inferior temporal, middle temporal, and banks of the superior temporal sulcus regions.

### Example 2

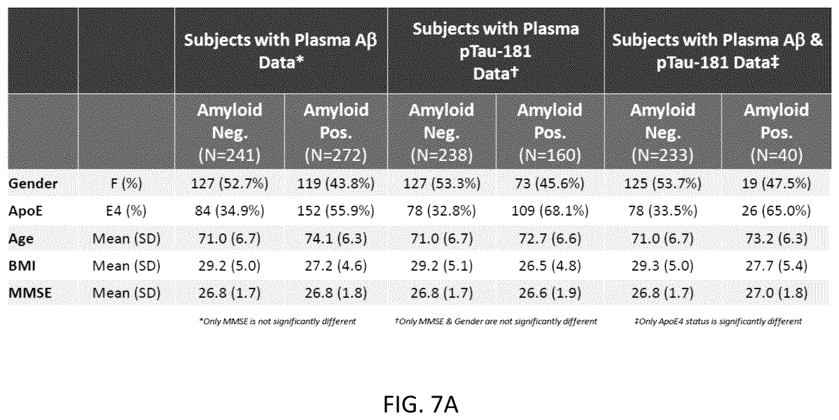

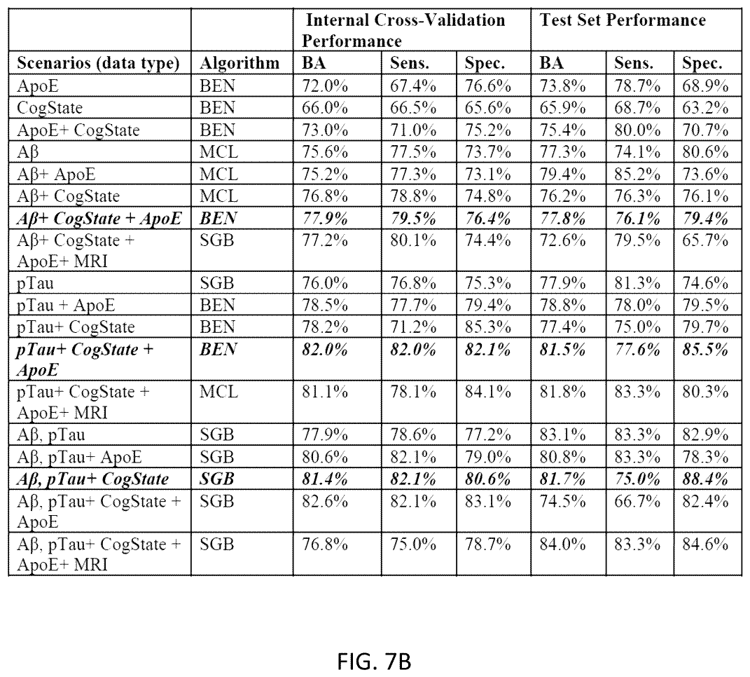

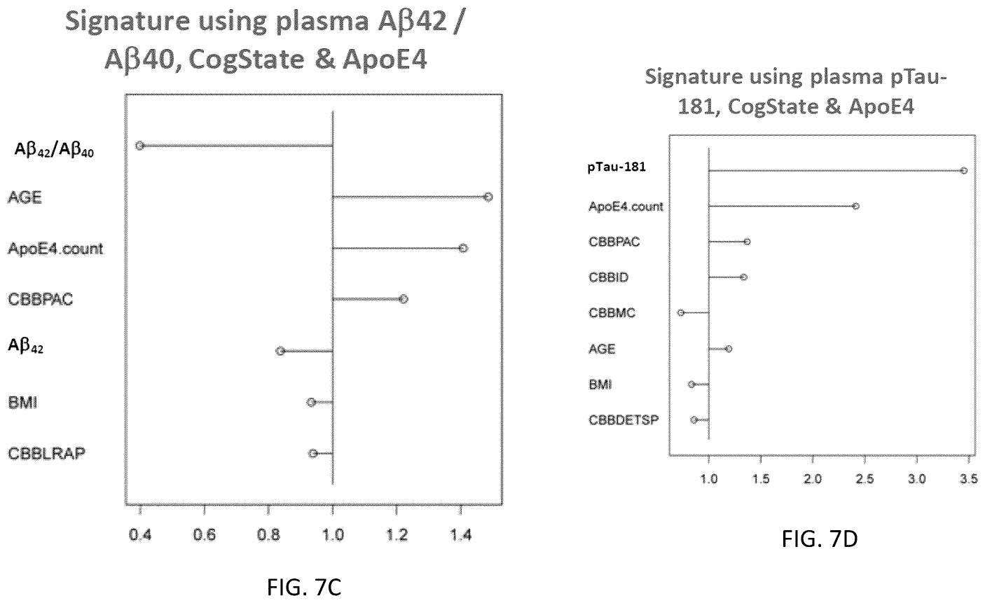

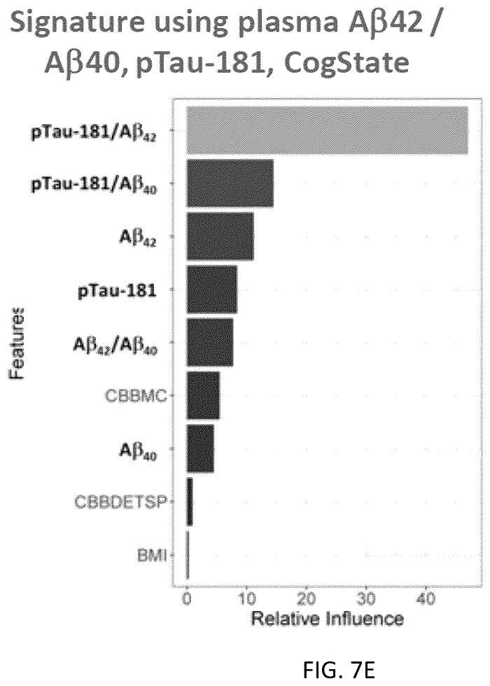

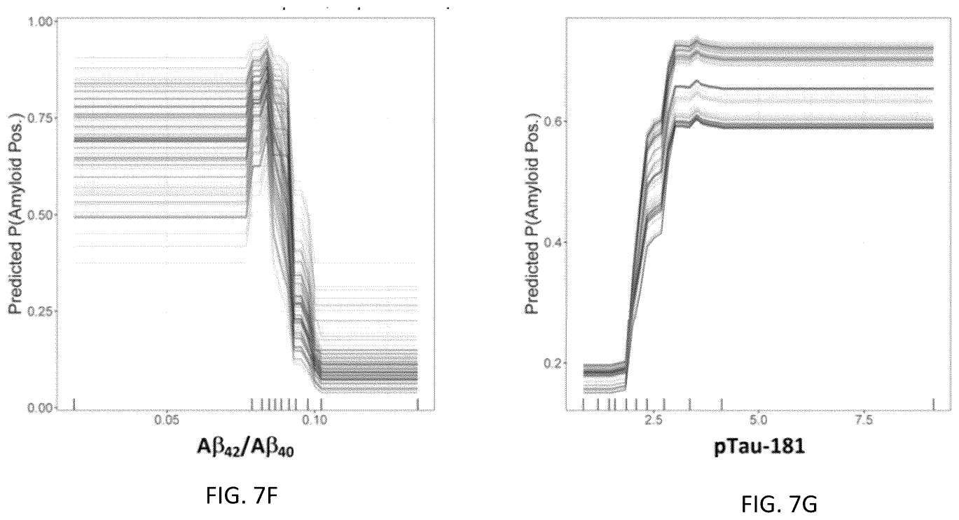

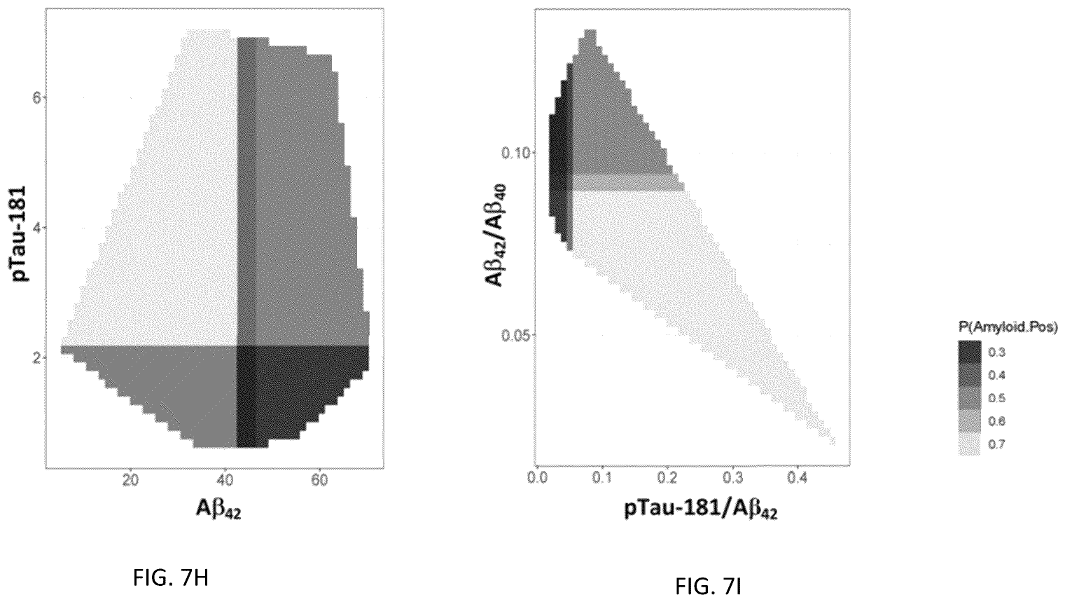

FIGS. 7A to 7I concern an investigation into prediction of brain amyloid-β (Aβ) status using blood-based tests. Predictive models were trained for brain Aβ detection using plasma biomarkers (Aβ42, Aβ40, and p-Tau181). The accuracy of such models was evaluated, as was the accuracy of models that additionally considered ApoE4 status, cognitive function measurements, and brain region measurements obtained from imaging data. The investigation demonstrated that predictive models configured to predict brain Aβ detection probabilities based on blood-based amyloid and tau signatures can be used as a screening tool for detecting brain amyloid burden in clinical trials and patient care.

In this investigation, Aβ42 and Aβ40 were measured using immunoprecipitation coupled with LC-MS/MS in plasma samples from 513 subjects at screening, and p-Tau181 was measured in plasma samples from 398 subjects (n=273 overlap) using Simoa Advantage V2 assay kit (immunoassay) by Quanterix. Over 90% of these subjects had mild cognitive impairment. Brain Aβ assessment was based on florbetaben-PET visual read for approximately 80% of the subjects, and the rest were assessed using florbetapir or flutemetamol.

FIG. 7A depicts the subject demographics and cognitive level measurement values for subjects with different combinations of input biomarker data. Some subjects had plasma Aβ measurements, some subjects had plasma p-Tau181 measurements, and some subjects had both types of measurement.

Linear regression-based machine-learning models (Monte-Carlo Logistic Lasso (MCL) and Bayesian Logistic Elastic Net (BEN)) and tree-based ensemble machine-learning models (regularized random forest (RRF) and stochastic gradient boosting machine (SGB)) were trained to detect brain Aβ. All models trained considered demographic inputs, while the improvement provided by ApoE4 status, cognitive function levels, and brain region MRI measurements was considered in some models.

Model performance was evaluated using 10 iterations of 10-fold stratified cross validation within a 70% training set. Further evaluation was performed using a 30% hold-out set. Models based on plasma markers were tested in the ADNI cohort, but cognitive levels could not be considered in this cohort due to limited data.

FIG. 7B depicts a summary of model performance for different predictive model inputs. Results for the best-performing model are depicted for each combination of inputs. In the table, BA refers to Balanced Accuracy (the average of sensitivity and specificity). The label Aβ refers to Aβ42/Aβ40 ratio. Each model includes only certain optimal features identified by hyperparameter tuning.

The best-performing predictive model using as inputs demographics, plasma Aβ42 and Aβ40 levels, cognitive measurements, and ApoE4 status achieved 78% accuracy for detecting brain Aβ, with the area under the receiver operating characteristic curve being 83.2%. The best-performing model using as inputs demographics and plasma Aβ42 and Aβ40 levels achieved an accuracy of 75.6%.

The best-performing predictive model using as inputs demographics, plasma p-Tau181 level, cognitive measurements, and ApoE4 status achieved 82% accuracy for detecting brain Aβ, with the area under the receiver operating characteristic curve being 87.4%. The best-performing model using as inputs demographics and plasma p-Tau181 level achieved an accuracy of 76%.

The best-performing predictive model using as inputs demographics, plasma Aβ42 and Aβ40 levels and plasma p-Tau181 level achieved 80.6% accuracy for detecting brain Aβ. The best-performing model using as inputs demographics, plasma Aβ42 and Aβ40 levels, plasma p-Tau181 level, and cognitive measurements achieved 82% accuracy for detecting brain Aβ with the area under the receiver operating characteristic curve being 87.3%.

When testing further in the independent ADNI cohort, the best-performing model achieved 75.7% accuracy using as inputs demographics and plasma Aβ42 and Aβ40 levels; the best-performing model achieved 75% accuracy using as inputs demographics and plasma p-Tau181 level, which improved to 78% when combined with plasma Aβ42 and Aβ40 levels.

FIGS. 7C to 7E each depict the input features with the greatest effect (in terms of odds ratio) on the output for three different Stochastic Gradient Boosting Machine (SGBM) predictive models. In these examples, certain cognitive measurements identified are CBBPAC (psychomotor attention), CBBLRAP (one-card learning accuracy), CBBID (identification), CBBMC (memory composite), and CBBDETSP (detection speed). FIG. 7C depicts the input features with the greatest effect (in terms of odds ratio) on the output for a model trained using plasma Aβ42 and Aβ40 levels, cognitive measurements, and ApoE4 status. FIG. 7D depicts the input features with the greatest effect (in terms of odds ratio) on the output for a model trained using plasma p-Tau181 level, cognitive measurements, and ApoE4 status. FIG. 7E depicts the input features with the greatest effect (in terms of odds ratio) on the output for a model trained using plasma Aβ42 and Aβ40 levels, plasma p-Tau181 level, and cognitive measurements.

FIG. 7F depicts a nonlinear sigmoidal relationship between plasma Aβ42 and Aβ40 levels and the predicted probability of brain Aβ detection for an SGB machine-learning model. Increasing Aβ42/Aβ40 ratios, above a threshold, resulted in a substantial decrease in the predicted probability of brain Aβ detection.

FIG. 7G depicts a nonlinear sigmoidal relationship between plasma p-Tau181 level and the predicted probability of brain Aβ detection for an SGB machine-learning model. Increasing plasma p-Tau181 level, above a threshold, resulted in a substantial increase in the predicted probability of brain Aβ detection.

FIGS. 7H and 7I depict heatmaps showing interactions between certain input values and a predicted likelihood of brain Aβ detection. FIG. 7H shows that the probability of brain Aβ detection increased with decreasing plasma Aβ42 levels and increasing plasma p-Tau181 levels. FIG. 7I shows that the probably of brain Aβ detection increased with decreasing Aβ42/Aβ40 ratios and increasing p-Tau181/Aβ42 ratios.

### Example 3

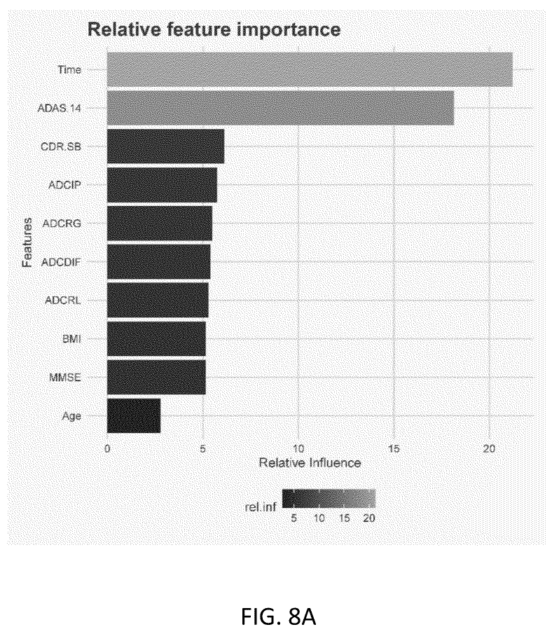

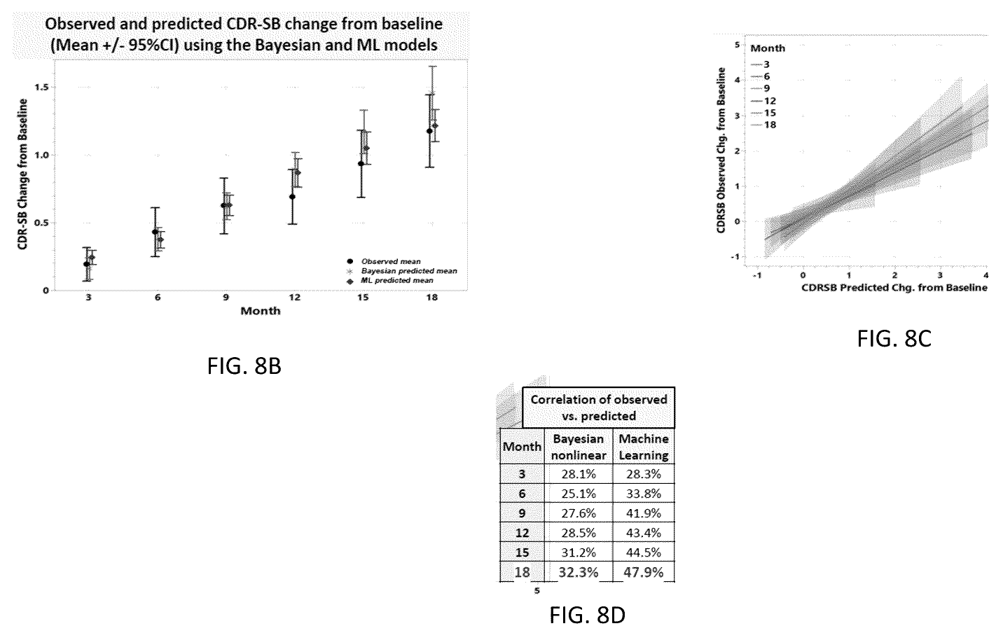

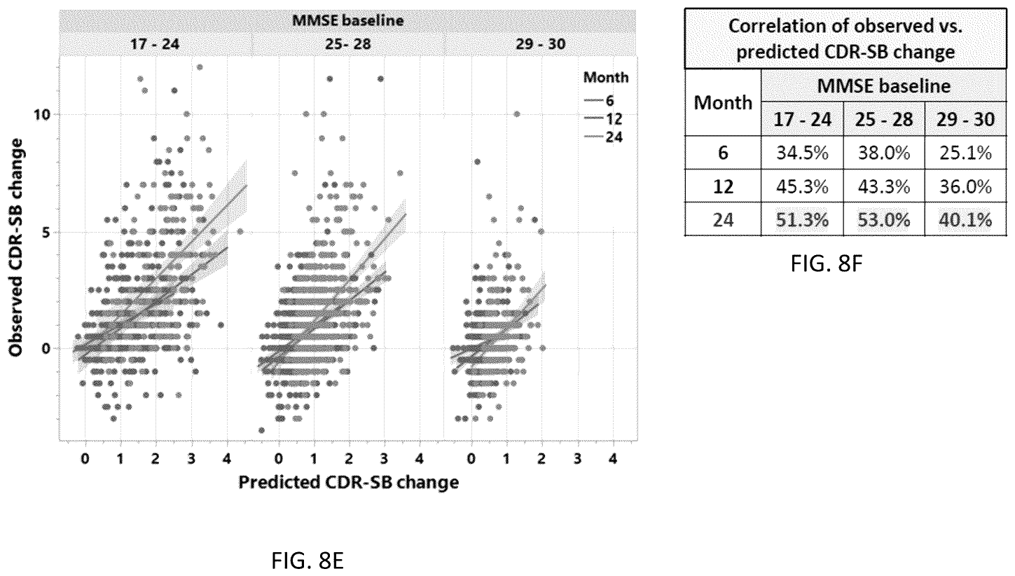

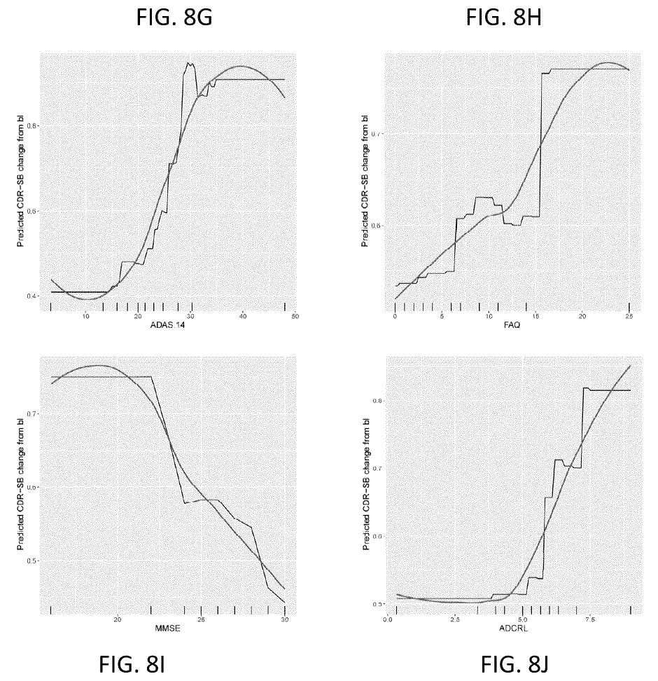

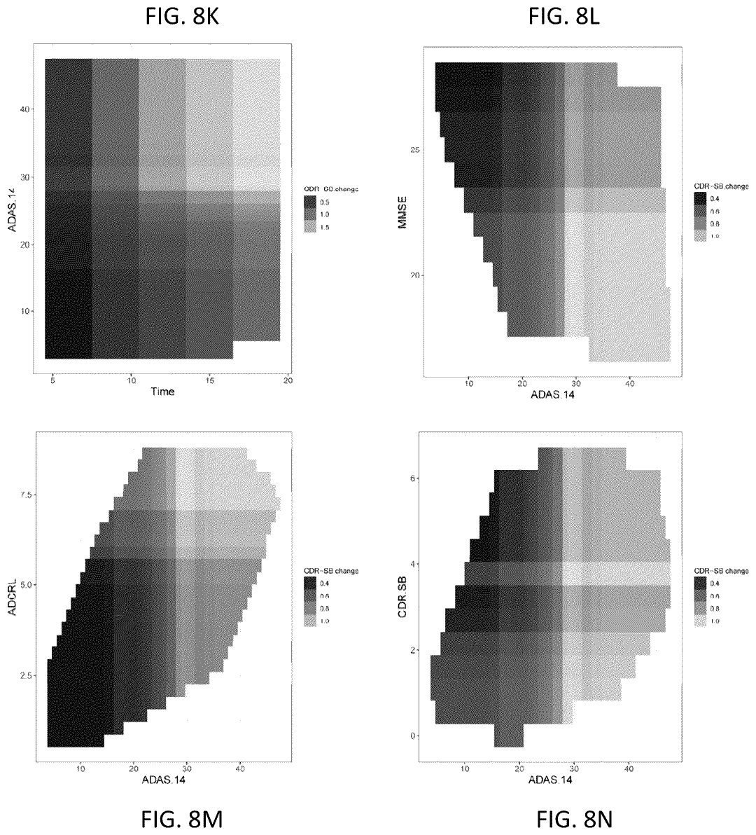

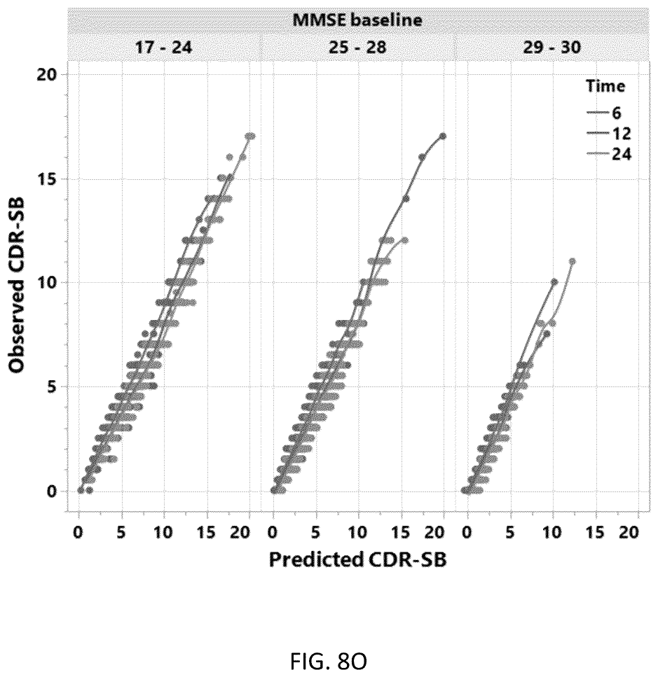

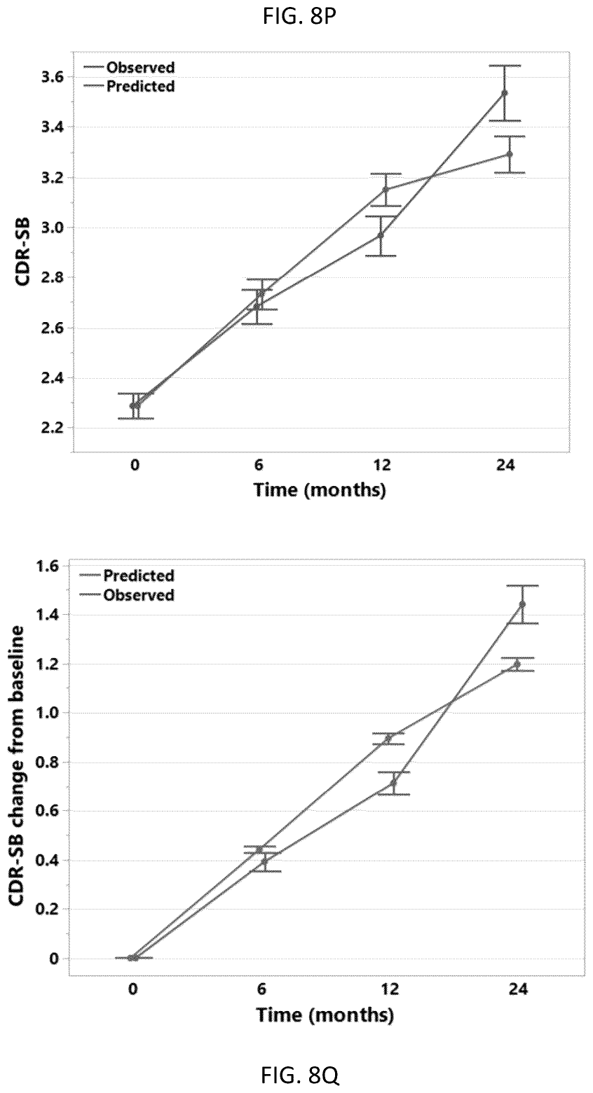

FIGS. 8A to 8Q concern an investigation into predicting the progression of cognitive impairment. The prediction used demographics, ApoE4 allelic count, and cognitive measurements obtained at baseline of each subject. Utilizing only screening/baseline data, the longitudinal cognitive trajectory of each subject was predicted using models trained on control data obtained from previously conducted clinical trials.

The prediction model was developed (trained) using historical control (placebo) data from mild cognitive impairment (MCI) and mild-AD subjects (n=955) from two clinical studies (NCT02956486 and NCT03036280). This model was constructed using the Stochastic Gradient Boosting Machine (SGBM) algorithm. The R package “gbm” was used to construct the model using this training data.

FIG. 8A depicts the relative importance of key input features to the trained model. In this example, ADCIP (ideational praxis), ADCRG (word recognition), ADCDIF (word finding difficulty) are components of the ADAS 13 score. Prediction performance of this model was evaluated in placebo subjects of another clinical study (NCT01767311) (n=231 subjects with non-missing input data). Spearman correlation of 43.2% was observed between the predicted versus observed CDR-SB change from baseline in month-18.

FIG. 8B depicts the observed progression of cognitive impairment as compared to the predicted progression of cognitive impairment for the two models, a Bayesian longitudinal mixed effects model and a stochastic gradient boosting model. The progression of cognitive impairment was measured as the CDR-SB change from baseline. The subjects were assessed at approximately 3, 6, 9, 12, 15, and 18 months.

FIG. 8C depicts the correlation between the observed and predicted cognitive impairment progression for individual subjects. As apparent from the figure, predicted and observed changes in cognitive performance from baseline were correlated.

FIG. 8D depicts the correlation between the observed and predicted values for the 3, 6, 9, 12, 15, and 18-month assessments for both the Bayesian longitudinal mixed effects model and the stochastic gradient boosting model.

The prediction model was retrained to use ADAS-13 cognitive measurements as opposed to ADAS-14 cognitive measurements. The retrained prediction model was used to predict the progression of cognitive impairment in the ADNI subject sample. FIG. 8E depicts the relationship between predicted cognitive impairment progression and observed cognitive impairment progression, as measured at 6, 12, and 24-month assessments. The observations were stratified by MMSE baseline value (17-24, 25-28, and 29-30). As depicted in FIG. 8F, the predicted and observed changes in cognitive measurements were correlated at the 6, 12, and 24-month assessments.

FIGS. 8G to 8J depict the relationship between four different predictive model inputs and the predicted change in cognitive measurements (e.g., predicted change in CDR-SB from baseline) for the stochastic gradient boosting model. As depicted in FIG. 8G, the predicted change in cognitive measurements exhibits an increasing sigmoidal response to increasing ADAS 14 score. As depicted in FIG. 8H, the predicted change in cognitive measurements exhibits an increasing response to increasing FAQ score. As depicted in FIG. 8I, the predicted change in cognitive measurements exhibits a decreasing response to increasing MMSE score. As depicted in FIG. 8J, the predicted change in cognitive measurements exhibits a thresholded and increasing response to increasing ADCRL subscore.

FIGS. 8K to 8N depict heatmaps showing interactions between certain input values and a predicted change in cognitive measurements (e.g., decrease in CDR-SB change from baseline) from the stochastic gradient boosting model. As depicted in FIG. 8K, the predicted change in cognitive measurements increased with increasing time and ADAS 14 score. As depicted in FIG. 8L, the predicted change in cognitive measurements increased with increasing ADAS 14 score and decreasing MMSE score. As depicted in FIG. 8M, the predicted change in cognitive measurements increased with increasing ADCRL subscore and increasing ADAS 14 score. As depicted in FIG. 8N, the predicted change in cognitive measurements increased as CDR-SB baseline score decreased and ADAS 14 score increased.

FIG. 8O depicts the relationship between the observed CDR-SB score and the predicted CDR-SB score for subjects in the ADNI study at 6, 12, and 24-month assessments, stratified by MMSE baseline. The observed and predicted CDR-SB scores show good alignment. FIG. 8P depicts the observed and predicted change from baseline in CDR-SB over time for subjects in the ADNI test dataset. FIG. 8Q depicts the observed and predicted CDR-SB values over time for subjects in the ADNI test dataset.

### Example 4

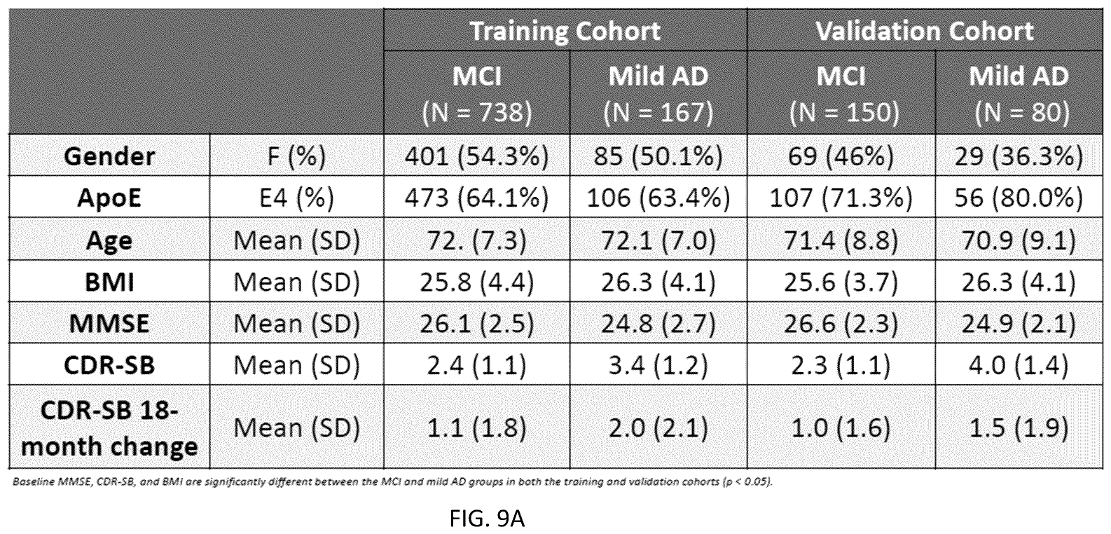

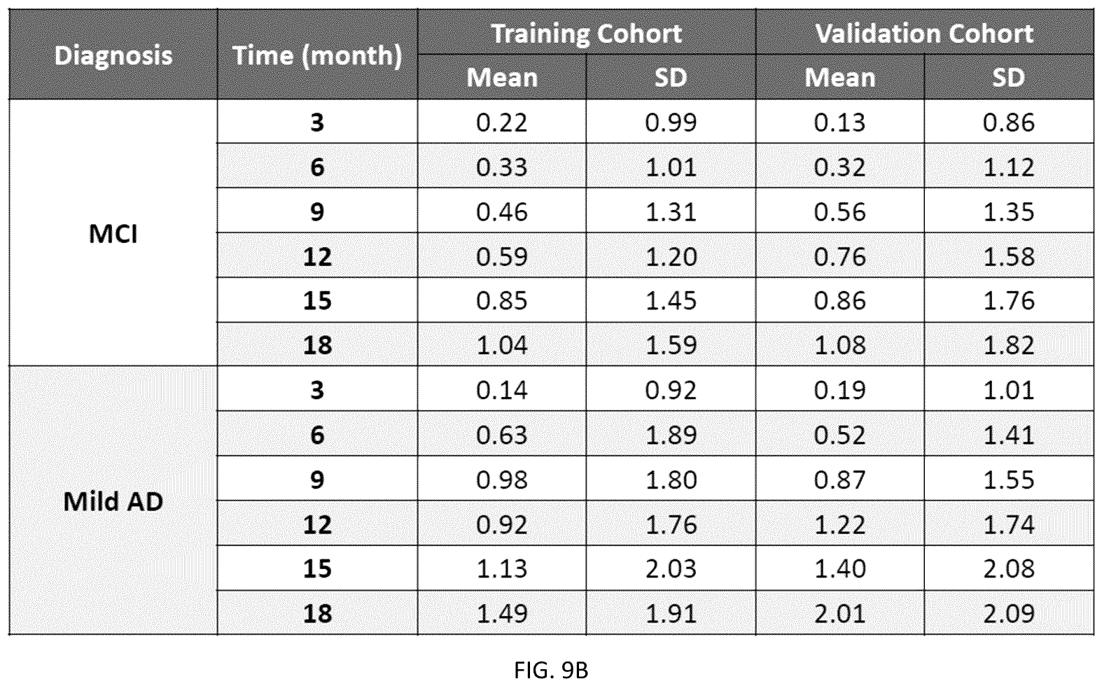

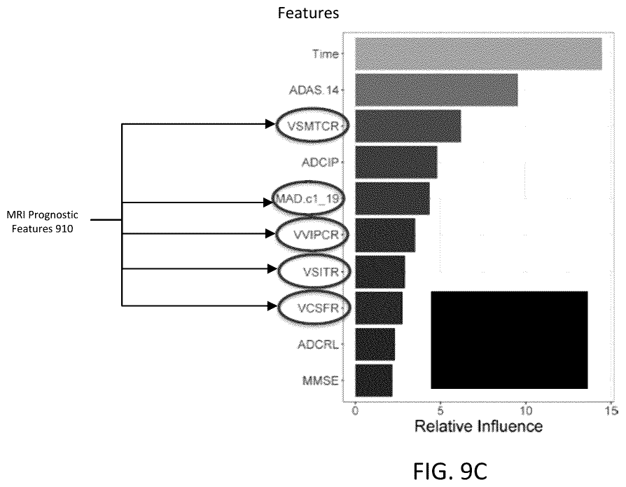

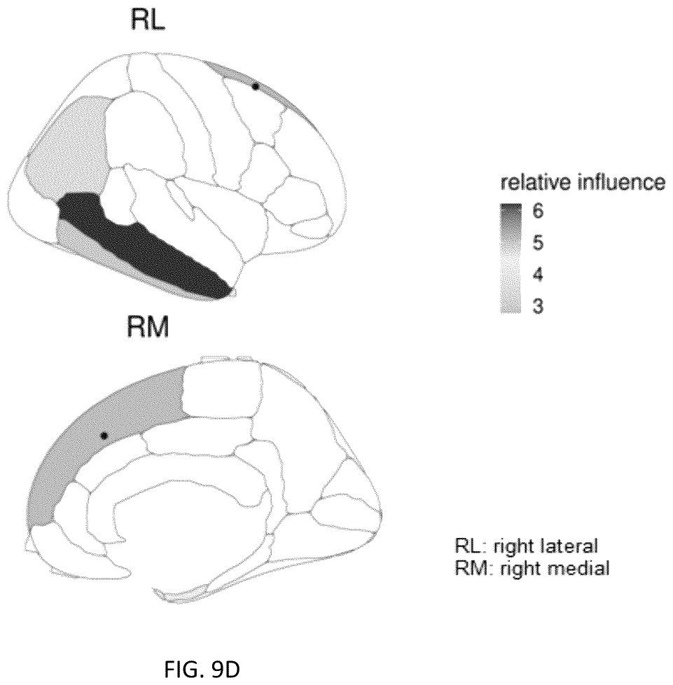

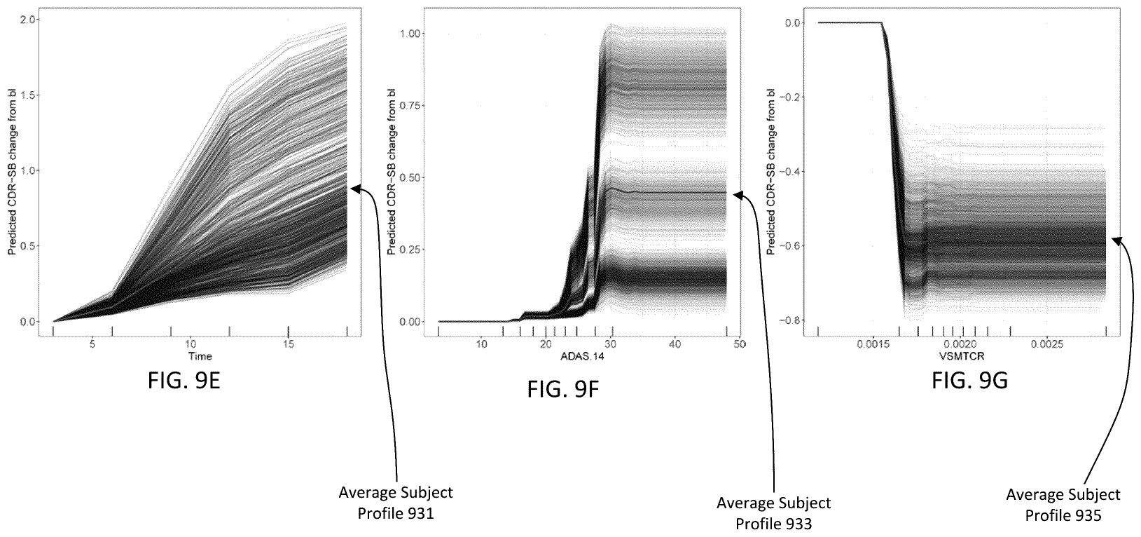

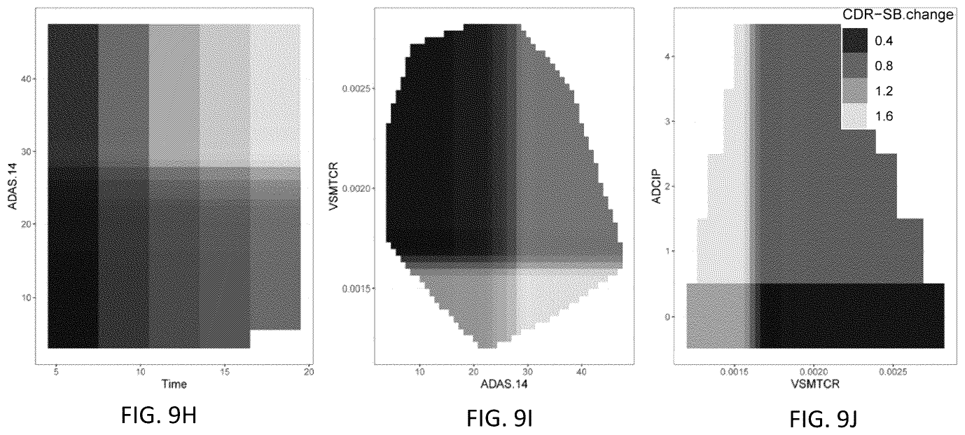

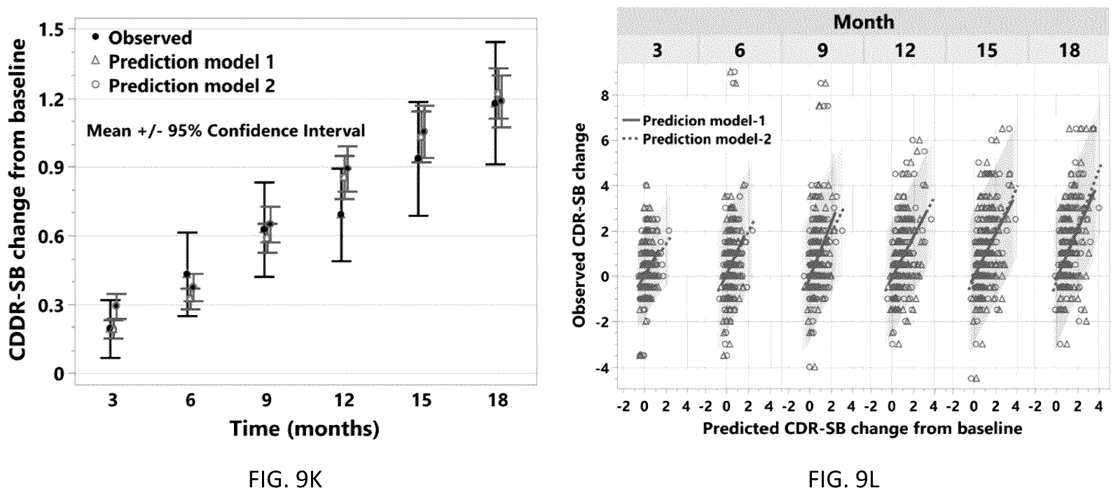

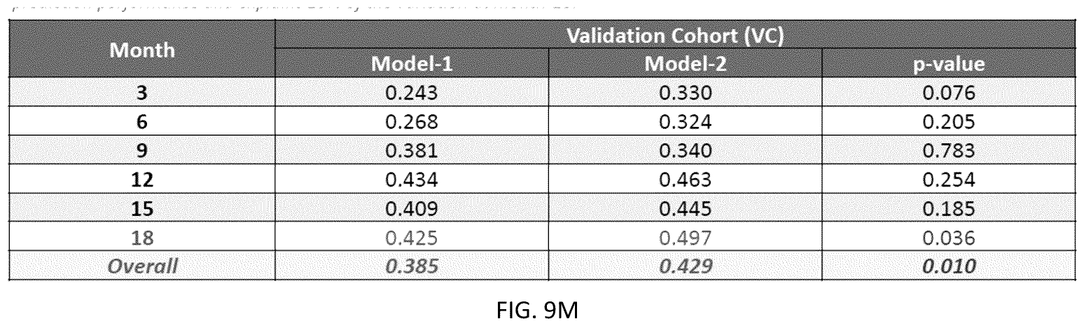

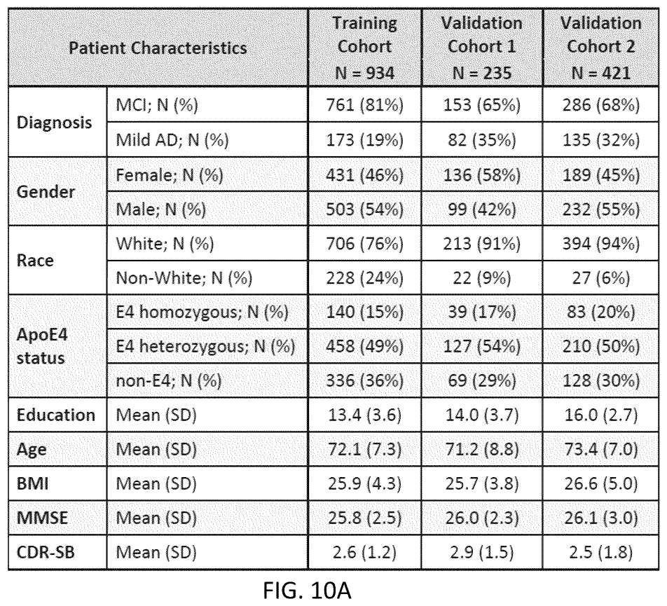

FIGS. 9A to 9M concern an investigation into predicting the progression of cognitive impairment in early Alzheimer's disease. Such predictions can be used for optimizing clinical studies and for patient monitoring. The investigation considered predictive models trained 1) using baseline patient demographics and clinical cognitive assessments, and 2) using baseline patient demographics, clinical cognitive assessments, and brain regional magnetic resonance imaging (MRI) measures (volume, surface area, and cortical thickness).

The investigation used a training cohort of 905 early AD subjects from two clinical trials of the same study trial and a validation cohort including 230 early AD subjects from another clinical trial. Cognitive performance (CDR-SB) was assessed at baseline and 3, 6, 9, 12, and 18-month assessments.

Brain MRI data (volume, surface area, and cortical thickness) in all three cohorts were generated for various brain regions of interest using the Desikan-Killiany atlas, resulting in 207 regional measures. Cortical thickness values are represented in millimeters (mm) and the volume (mm³) and surface area (mm²) were normalized by the intra cranial volume to reduce inter subject variability. Hubs and modules were identified from among brain regions using MEGENA.

Predictive models were trained using baseline cognitive measurements and demographics for the training cohort. The predictive models included regularized random forests, support vector machines, Bayesian lasso regression models and stochastic gradient boosting models. Additional predictive models were then trained using baseline cognitive measurements, demographics, and the identified hubs and modules.

Prediction performance of the predictive models was first evaluated within the training cohort using 10 iterations of 10-fold cross-validation. The performance of the top-performing predictive model was then evaluated in first validation cohort via the Spearman correlation between the observed versus predicted cognitive trajectory. Results are reported for the stochastic gradient boosting model, as it achieved the best performance among the models tested.

FIG. 9A depicts a demographic summary of the training cohort and the first validation cohort. FIG. 9B depicts summary of the change in CDR-SB score for the training cohort and first validation cohort, broken out be diagnosis of MCI or Mild AD at baseline.

FIG. 8A depicts the relative influence of the top-ten most important inputs to the best-performing predictive model, when restricting inputs to baseline cognitive measurements and demographics. FIG. 9C depicts the relative influence of the relative influence of the top-ten most important inputs to the best-performing predictive model, when the input dataset for training the predictive model additionally included hubs and modules (e.g., MRI prognostic features 910). FIG. 9D depicts heatmaps of the key hubs and modules referenced in FIG. 9C. The key hubs and regions included the VSMTCR (the middle temporal area), the VVIPCR (the inferior parietal cortical volume), the VSITR (the inferior temporal cortical area), the VCSFR (the superior frontal cortical thickness), and SBN module (the SBN module that includes inferior parietal, inferior temporal, middle temporal, and cortical areas around superior temporal sulcus).

FIGS. 9E to 9G depict the impact of certain inputs in predicting cognitive impairment progression. These figures show individual conditional expectation (ICE) profiles for each subject and for the average subject (e.g., average subject 931, 933, and 935). The vertical axis represents the normalized predicted change in CDR-SB (normalizing for the decline at the minimum value of the predictor). Inter-subject heterogeneity in these ICE profiles was due to the strong interaction between the predictors. These nonlinear relationships and interactions were accounted for by the stochastic gradient boosting algorithm without prior assumptions.

FIGS. 9H to 9J depict interaction prediction profiles for certain inputs in predicting cognitive impairment progression. The interaction prediction profiles reveal dependences between these inputs. FIGS. 9H to 9J show that, while all subjects exhibited greater changes in CDR-SB scores over time, subjects with high ADAS-14 scores at baseline tended to exhibit greater changes in CDR-SB scores over time. Furthermore, among subjects with high ADAS-14 scores at baseline, subjects with lower scores in certain components of the ADAS-14 assessment (e.g., those particularly weak in word recognition) experienced faster cognitive impairment progression. Similarly, subjects with a lower middle temporal cortical area score (e.g., VSMTCR score) experienced faster cognitive impairment progression if they were more cognitively impaired at baseline (i.e., higher baseline ADAS-14 score).

FIGS. 9K to 9L depict comparisons between observed and predicted cognitive impairment for two predictive models using the first validation cohort. FIG. 9K depicts predicted mean cognitive impairment using two prediction models: a first prediction model trained using baseline demographics and cognitive measurements (model-1) and a second prediction model trained using baseline demographics, cognitive measurements, and baseline node and hubs (model-2). As can be observed in FIG. 9K, the predicted cognitive impairment tracked well with the mean observed cognitive impairment for both models. FIG. 9L depicts the relationships between observed and predicted cognitive impairment for individual subjects, as measured at the 3, 6, 9, 12, 15, and 18-month assessments, for both models. The observed and predicted cognitive impairment values correlated for the subjects in the validation cohort.

FIG. 9M depicts the spearman rank correlation of the observed cognitive impairment versus the predicted cognitive impairment for subjects in the first validation cohort. Cognitive impairment was predicted using the two predictive models of FIGS. 9K and 9L. An additional column includes p-values comparing the correlations. The predicted 18-month cognitive impairment achieved a correlation of 0.425 with the observed 18-month assessment (explaining 18.1% of the variation) when using the first predictive model. Adding baseline nodes and hubs in the second model significantly improved the overall prediction performance and explained 25% of the variation at the 18-month assessment.

### Example 5

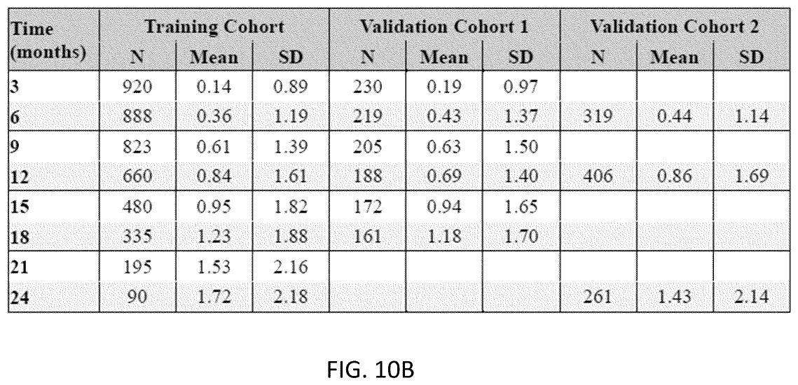

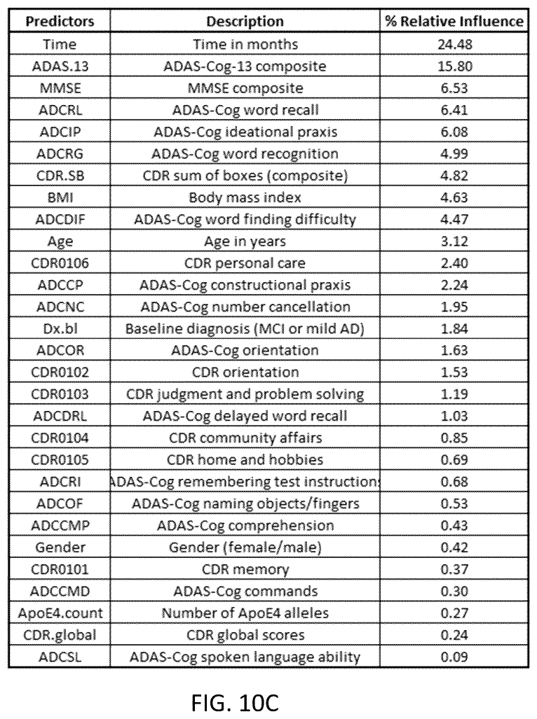

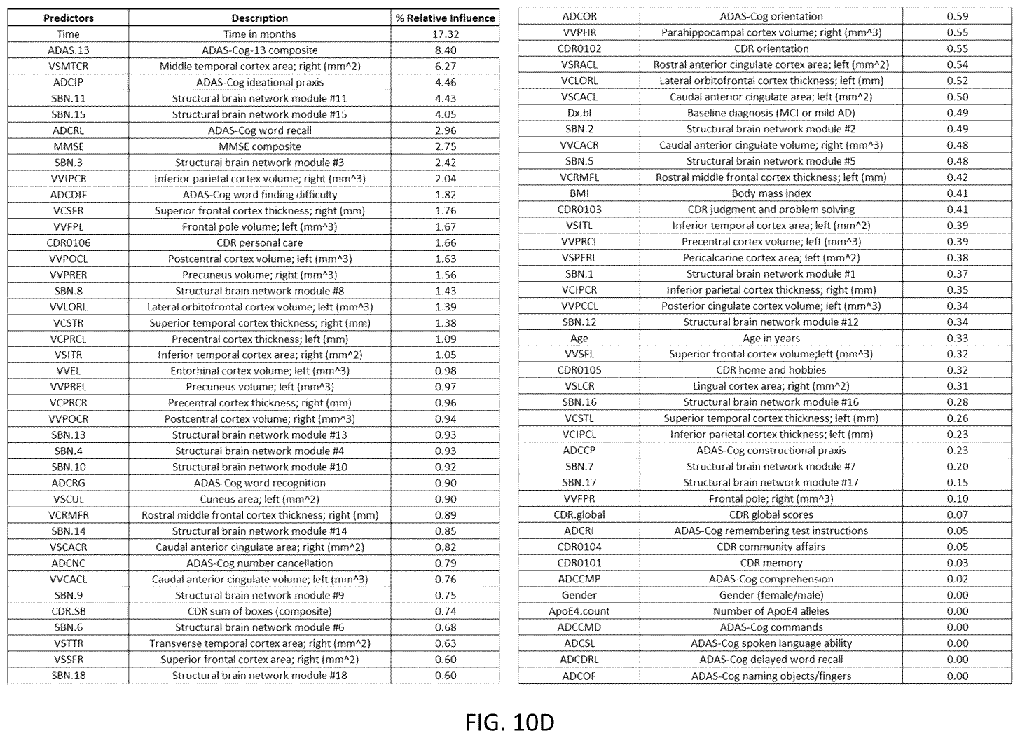

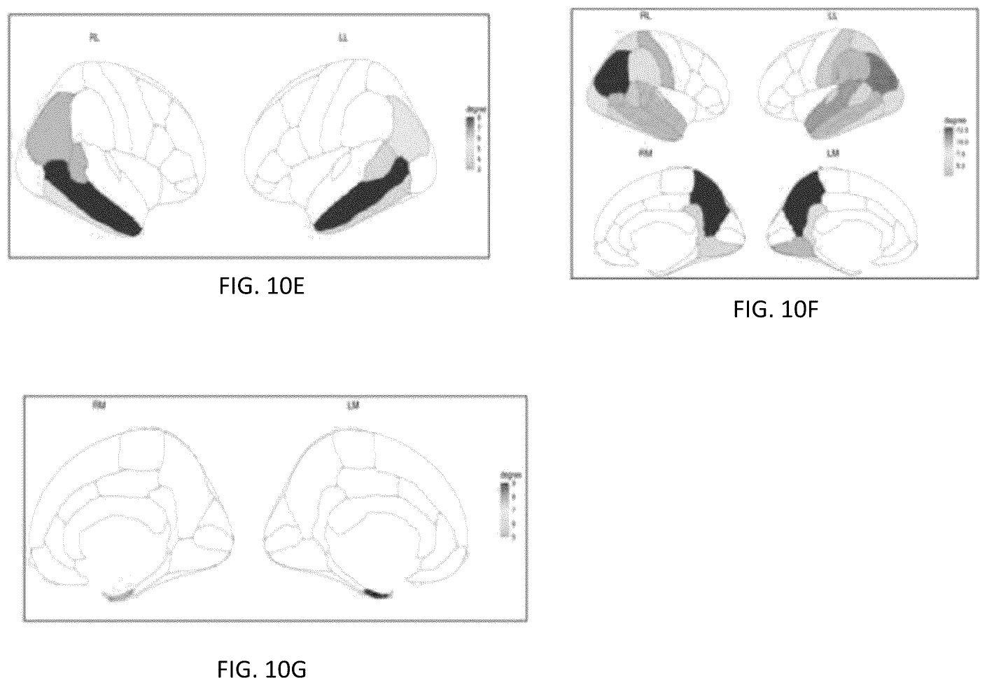

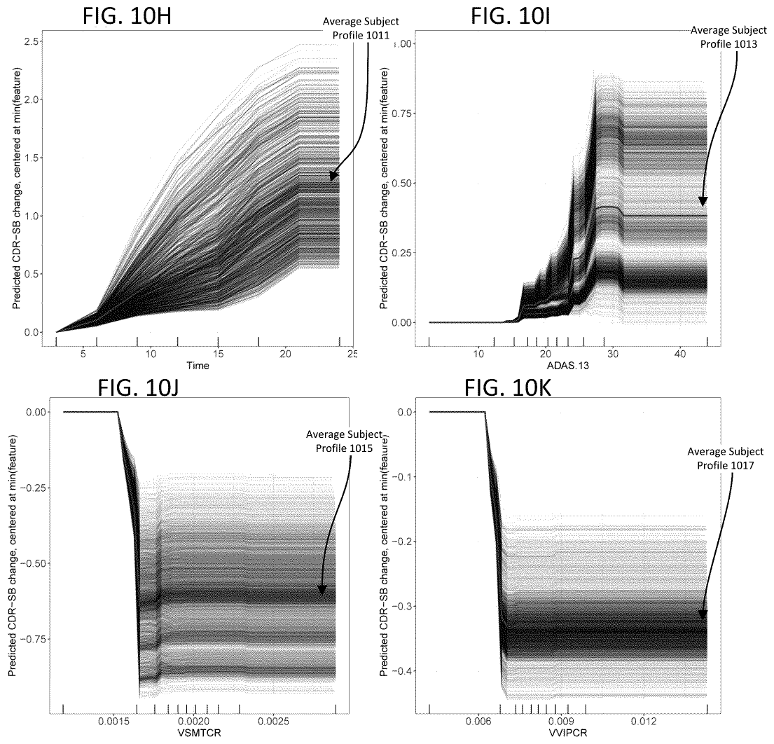

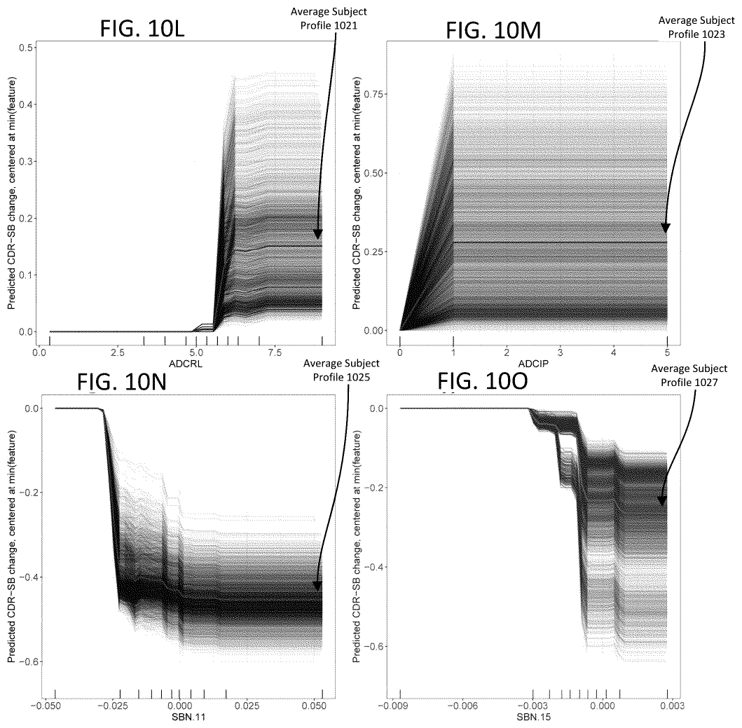

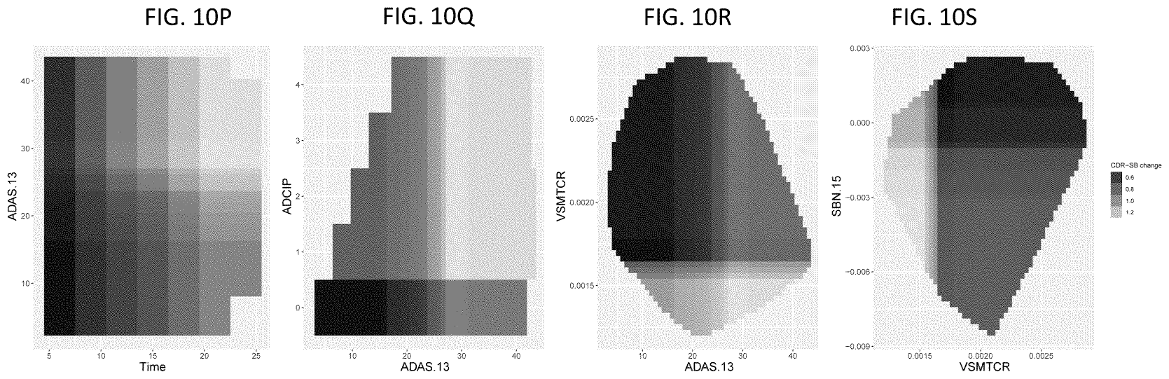

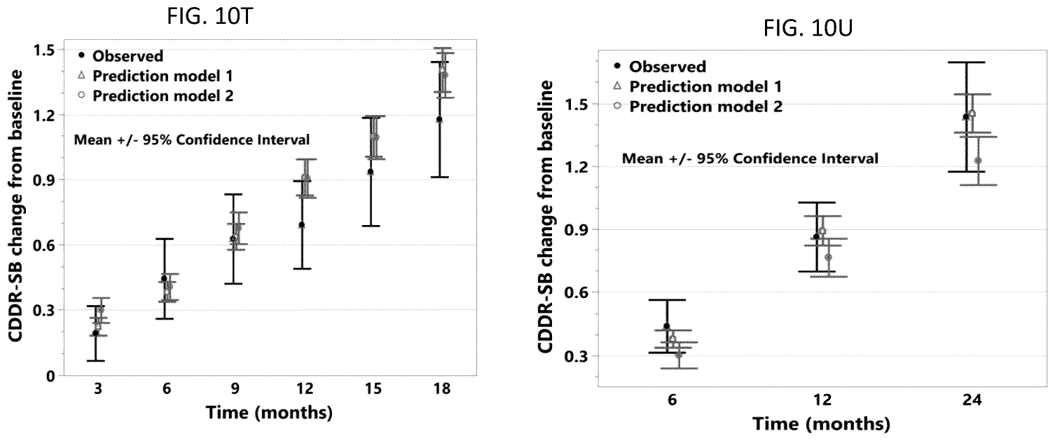

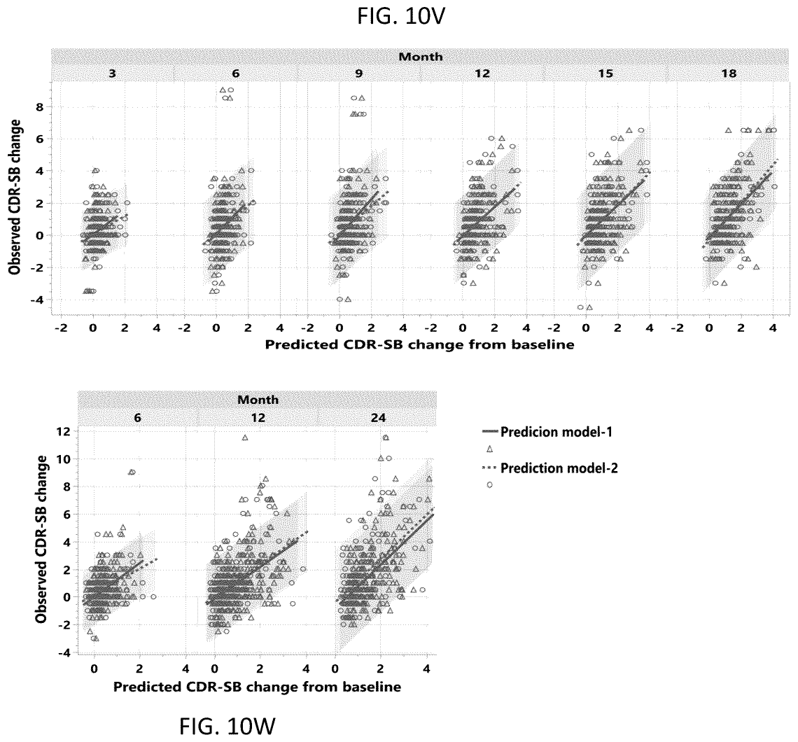

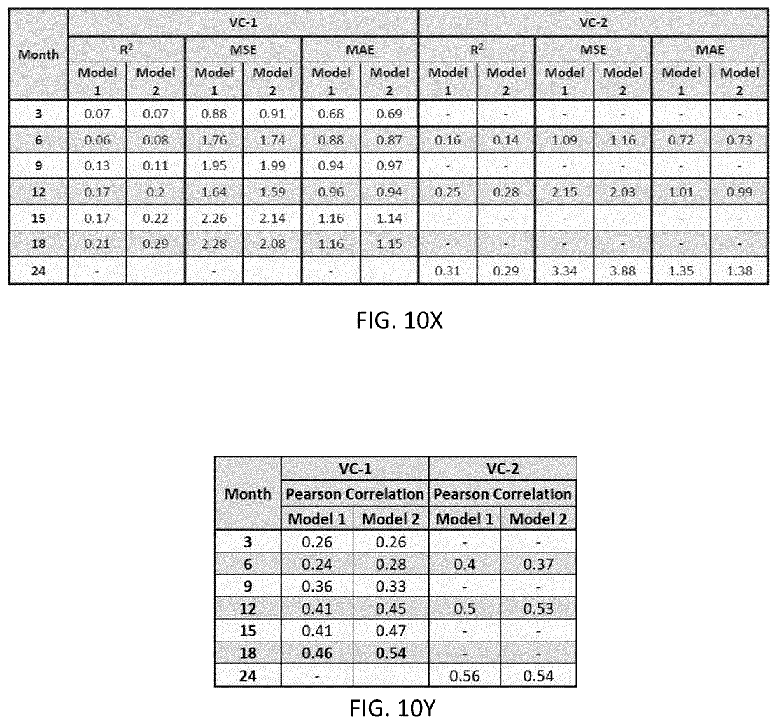

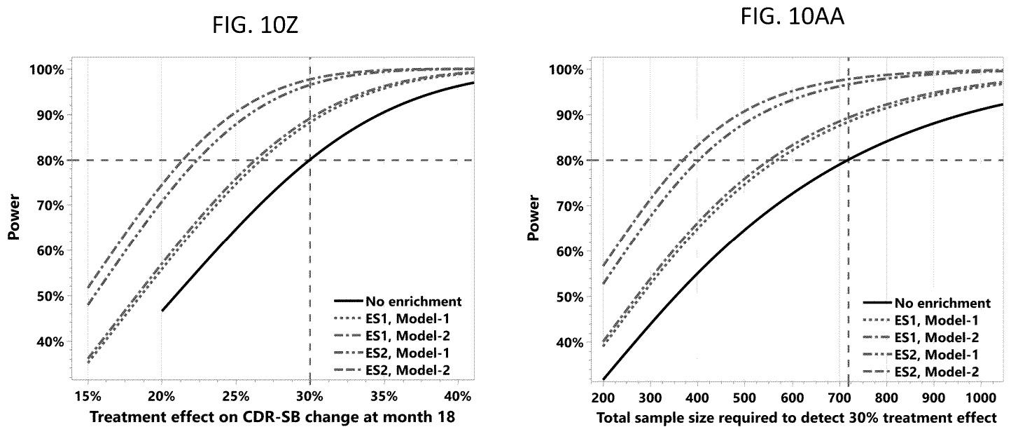

FIGS. 10A to 10W concern an investigation into predicting progression of cognitive impairment in early Alzheimer's disease. In this investigation, gradient boosting predictive models were trained to predict cognitive impairment progression in A+ early AD subjects over an 18 to 24-month duration. The predictive models were trained using a training cohort (TC, n=934). The training cohort included historical placebo subject data from two clinical trials (NCT02956486 and NCT03036280), both part of the same phase-3 program. Over 81% of the placebo subjects in the training cohort had mild cognitive impairment due to AD (MCI), and the rest had mild AD. The trained predictive models were evaluated in two validation cohorts (VC-1, n=235; VC-2, n=421). The first validation cohort (VC-1) included A+ early AD patients from the placebo arm of an 18-month clinical study (NCT01767311). The second validation cohort (VC-2) included A+ subjects diagnosed as either MCI or AD with at least one year of clinical follow-up and the relevant clinical and MRI assessments from the ADNI-2 and ADNI-3 phases of the ADNI database. The best-performing predictive model using cognitive measures and demographics achieved R² of 0.21 and 0.31 for predicting 2-year cognitive decline in VC-1 and VC-2, respectively. The best-performing predictive model using cognitive measures, demographics, and MRI features achieved R² of 0.29 in VC-1, which employed the same preprocessing pipeline as TC. Utilizing these model-based predictions for clinical trial enrichment reduced the required sample size by 20% to 49%.

Cognitive impairment was defined in terms of the change from baseline in CDR-SB. Cognitive measurements included as inputs to the predictive models included the composite endpoints: Mini-Mental State Examination (MMSE), Alzheimer's Disease Assessment Scale-Cognitive Subscale (ADAS-Cog-13), CDR-SB, and all their sub-scores. In the training cohort, cognitive measurements were assessed at months 3, 6, 9, 12, 15, 18, 21, and 24. The clinical follow-up times considered for evaluating the prediction models in the first and second validation cohorts were months 3, 6, 9, 12, 15, 18, and months 6, 12, and 24, respectively.

All subjects in TC and VC-1 received a 3.0 Tesla (T) structural MRI at baseline. Approximately 75% of subjects in VC-2 received 1.5 T and the rest received 3 T MRI. Brain MRI data (volume, area, and cortical thickness) in all three cohorts were generated for various brain regions of interest using the Desikan-Killiany atlas, resulting in 207 regional measures. Cortical thickness values are represented in millimeters (mm). The volume (mm³) and area (mm²) were normalized (divided) by the intra-cranial volume to reduce inter-subject variability and account for variance due to head size within each cohort.

FIG. 10A depicts the demographic and clinical data characteristics of the training and validation cohorts. All the demographic and clinical characteristics were significantly different (p<0.05) between the cohorts. The training cohort had a significantly greater proportion of MCI and ApoE4-positive subjects. The first validation cohort (VC-1) had a greater proportion of males. Subjects in the second validation cohort (VC-2) were older and had higher body mass index (BMI). These differences among early AD subjects across different clinical trial and observational cohorts helped to provide a more generalizable assessment of the performance of the prediction models between the training and validation cohorts.

FIG. 10B depicts the longitudinal change in CDR-SB at each assessment. The mean and standard deviation (SD) of CDR-SB change from baseline in the training and validation cohorts are depicted for each time point, along with the number of subjects available for assessment.

Modules and hubs were derived using 207 MRI regional measures (volume, area, and cortical thickness) in the training cohort using MEGENA. As described herein, this process entails the calculation of the correlation of MRI measures across all pairs of regions. Regions with significant correlations were embedded on a spherical surface and representative edges (regions that are correlated with multiple other regions) were extracted to create planar-filtered networks. Finally, a hierarchy of network modules was constructed by recursively clustering the regional measures with coherent structures into network modules. This resulted in a total of 18 SBN modules (labeled as SBN.1 to SBN.18) and 45 hub regional measures. Some regions were present in more than one network module depending on the nature of correlations between the neighboring regions. The regional measures in each of the SBN modules were aggregated into a single composite eigenvalue for each subject using MEGENA. Subsequent prediction modeling efforts focused only on these 18 SBN modules and 45 hub regional measures.

A predictive model for predicting longitudinal cognitive trajectory for each subject was trained using baseline cognitive function data, demographic data, genomic data, and measurement time of cognitive function as predictors. The predictive model was a stochastic gradient boosting model. To train the model, up to 1000 decision trees were assembled with up to 3-way interactions among predictors. The ranking and relative influence of each predictor was derived by assessing the reduction in the mean squared error each time the predictor was used as a root node to split the decision trees in the SGBM algorithm, and these were then normalized to range from 0 to 100%. Insights into the relationships between predictors and outcomes and the interaction between predictors were derived via individual conditional expectation (ICE) profiles and partial dependence plots of the prediction profiles.

Model performance was evaluated using 10 iterations of 10-fold cross-validation within the TC. Subsequently, the models were evaluated in VC-1 and VC-2. This evaluation involved measuring the coefficient of determination (R²), mean squared error (MSE), and mean absolute error for observed versus predicted cognitive decline (CDR-SB change from baseline) at each time point.

FIG. 10C depicts the input variables ranked by relative importance to the measured output variable for the first model. Another such predictive model was trained using the above inputs and additionally the hubs and modules. FIG. 10D depicts the input variables ranked by relative importance to the measured output variable for the second model. In addition to Time, the key baseline clinical predictors in these models were ADAS-13 score, MMSE, word recall and recognition, ideational praxis, CDR-SB, and word-finding difficulty, along with BMI and age. Ideational praxis refers to the ability to perform multi-level tasks, for example, the sequence of steps needed for brushing teeth. Some of the key MRI-based predictors include the hub measures of middle temporal cortical area and inferior parietal cortical volume, along with the measures for i) the module comprising the inferior parietal gyri, inferior temporal gyri, middle temporal gyri, and banks of the superior temporal sulci (FIG. 10E), ii) the module comprising the entorhinal cortices and temporal poles (FIG. 10G), and iii) the module comprising the superior parietal gyri, precunei, isthmus of the cingulate gyri, lateral occipital gyri, postcentral gyri, supramarginal gyri, superior temporal gyri, fusiform gyri, lingual gyri, transverse temporal gyri plus the regions depicted in FIG. 10E (FIG. 10F).

FIGS. 10H to 10O depict the relationship between certain baseline inputs and the predicted cognitive impairment progression for two prediction models at the individual subject level and an average subject level using individual conditional expectation (ICE) profiles. The ICE profiles were generated by plotting the individual and average predicted outcomes (e.g., thus generating average subject profiles 1011, 1013, 1015, 1017, 1021, 1023, 1025, 1027) for different values of a baseline input while holding the values of other inputs constant. As depicted in FIGS. 10H to 10O, the ICE prediction profiles reveal strong sigmoidal-like nonlinear relationships between each baseline input and cognitive impairment progression. These relationships exhibit floor and ceiling effects and an intermediate region of linear impact. The prediction profile of each subject was centered by subtracting from the predicted CDR-SB change corresponding to the lowest value of the predictor. The gradient boosting predictive models allowed for the relationships and inflection nodes of the predictors to be modeled without prior specifications or assumptions.

FIGS. 10P to 10S depict interaction profiles between certain baseline inputs and the predicted cognitive impairment. FIG. 10P shows greater cognitive impairment over time in subjects with higher baseline ADAS-13 score. FIG. 10Q shows greater cognitive impairment in subjects with high ADAS-13 score and worse ideational praxis (ADCIP) at baseline. FIG. 10R shows greater cognitive impairment in subjects with high ADAS-13 score and lower middle temporal cortical area (VSMTCR). FIG. 10S shows greater cognitive impairment in subjects with lower middle temporal cortical area (VSMTCR) and lower area, volume, or thickness in the entorhinal cortex and temporal pole (SBN.15).

FIGS. 10T and 10U depict mean and 95% confidence interval of the observed and predicted CDR-SB change from baseline for the two models on both validation datasets. The CDR-SB change from baseline was predicted using the models based on the baseline clinical features (model-1) alone and with the addition of hubs and modules (model-2). FIG. 10T depicts the observed and predicted CDR-SB change from baseline for validation cohort 1. FIG. 10U depicts the observed and predicted CDR-SB change from baseline for validation cohort 2. The average predicted cognitive impairment tracked well and was not significantly different from the average observed cognitive impairment in both the validation cohorts across all time points.

FIGS. 10V and 10W depict the observed versus predicted CDR-SB change from baseline for individual subjects at each time point for the two models for both validation datasets. The CDR-SB change from baseline was predicted using the models based on the baseline clinical features (model-1) alone and with the addition of hubs and modules (model-2) along with 95% prediction intervals. FIG. 10V depicts the observed and predicted CDR-SB change from baseline for validation cohort 1. FIG. 10W depicts the observed and predicted CDR-SB change from baseline for validation cohort 2. Predictions of cognitive impairment of individual subjects from the two models were significantly correlated (p<0.001) with the observed cognitive impairment.

FIGS. 10X and 10Y depict the effect of using the disclosed predictive models for clinical trial enrichment, consistent with disclosed embodiments. As may be appreciated, patients may be selected for inclusion in a clinical trial based on predicted degree of cognitive impairment. Such patients may have a greater need for treatment. Furthermore, detectable treatment effect, study size, and study power can be improved by screening or selecting patients for inclusion in a clinical trial based on predicted degree of cognitive impairment.

In this investigation, 500 clinical trials were simulated via the bootstrap approach (sampling with replacement) based on the data from the placebo arm of the clinical trial used for VC-1, with a 1:1 random allocation of active treatment and placebo. The clinical trial duration was set at 18 months. The treatment effect, defined as the difference in the change from baseline in CDR-SB between the treatment and placebo groups at month 18, was set at 30%. The impact of selecting only patients with predicted 18-month CDR-SB change of at least 0.5 and 1 (enrichment scenarios 1 and 2 respectively) was then evaluated for each simulated clinical trial by comparing the sample size requirement and power between the non-enriched and enriched clinical trials for these different enrichment scenarios. The sample size evaluations were based on the two-sample t-test.

The impact on the sample size reduction and power increase is shown in FIGS. 10X and 10Y when enriching the clinical trial for subjects with predicted 18-month CDR-SB satisfying two thresholds (ES1 at least 0.5, and ES2 at least 1). Approximately 88% and 65% of the clinical trial subjects used in VC-1 met these two criteria. Results for these two enrichment scenarios were determined with two predictive models. A first predictive model that used only baseline cognitive measurements and demographics (model-1) and a second predictive model that used baseline cognitive measurements, demographics, and image data (e.g., hubs and modules) (model-2).

A clinical trial that did not use a trained predictive model for patient selection or screening would require a total sample size of 718 subjects (359 per group) to detect a 30% treatment effect with respect to the change from baseline in CDR-SB at month 18 with 80% power.

FIGS. 10X and 10Y present breakdowns of predictive model performance. FIG. 10X depicts a summary of prediction performance for two models in the two validation cohorts (VC-1 and VC-2). The first predictive model used cognitive measures and demographics (model-1). The second predictive model used cognitive measures, demographics, and image data (e.g., hubs and modules) (model-2). The prediction measures include coefficient of determination (R²), mean squared error (MSE), and mean absolute error (MAE) of the predicted versus observed clinical decline (CDR-SB change from baseline). Model-1 achieved R² of 0.21 and 0.31, along with Mean Squared Error (MSE) values of 2.28 and 3.34, and Mean Absolute Error (MAE) values of 1.16 and 1.35 for predicting cognitive decline at 18 months and 24 months in VC-1 and VC-2, respectively. Model-1 predictions were mostly on par with model-2, except at later time points with respect to R² and MSE in VC-1, which employed the same image processing pipeline as the training cohort (TC). In VC-1, model-2 achieved R² of 0.29 and MSE of 2.08.

FIG. 10Y depicts the Pearson correlation coefficients of the predicted versus observed cognitive decline (CDR-SB change from baseline) for two predictive models on the two validation cohorts (VC-1 and VC-2). The first predictive model used cognitive measures and demographics (model-1). The second predictive model used cognitive measures, demographics, and image data (e.g., hubs and modules) (model-2).

As depicted in FIG. 10Z, using the first model increased power to 88.3% and 96.5% respectively for the two enrichment scenarios. Fixing the power at 80%, use of the first model improved the ability to detect the treatment effect from 30% to 26.7% and 22.3% respectively. As depicted in FIG. 10AA, using the first model reduced the total sample size required to detect a 30% treatment effect from 718 to 568 and 398 (20.9% and 44.6% reduction) respectively for the two enrichment scenarios. Using the second model improved these numbers: for the two enrichment scenarios, the power increased to 89.2% and 97.6% respectively, and the minimum treatment effect that could be detected with 80% power improved from 30% to 26.3% and 21.3% (FIG. 10Z). The total sample size required to detect a 30% treatment effect was reduced from 718 to 552 and 364 (23.2% and 49.4% reduction) respectively for the two enrichment scenarios using model-2 predictions (FIG. 10AA).

Screening patients using a predictive model may decrease the number of patients that require screening. For the VC-1 clinical trial population and using the prediction model-2, approximately 89% and 62% met the ES1 and ES2 enrichment criteria, respectively. The total sample size required to detect a 30% treatment effect reduced from 718 to 552 and 364 (23.2% and 49.4% reduction), respectively. Therefore, instead of screening 718 subjects, only 620 subjects (552 divided by 0.89) need be screened in ES1 and 587 subjects (364 divided by 0.62) in ES2.

Thus, in addition to significant reductions in sample size requirements and an increase in power, screening patients using a trained predictive model as described herein may enable screening of 13.6% and 18.2% fewer subjects (e.g., as compared to the no enrichment strategy). More importantly, there may be other practical benefits/need for such enrichment strategies in clinical trials, for example, if the candidate treatment is expected to benefit only subjects that are likely to experience mild to moderate cognitive decline.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware, but systems and methods consistent with the present disclosure can be implemented with hardware and software. In addition, while certain components have been described as being coupled to one another, such components may be integrated with one another or distributed in any suitable fashion.

Embodiments herein include systems, methods, and tangible non-transitory computer-readable media. The methods may be executed, at least in part for example, by at least one processor that receives instructions from a tangible non-transitory computer-readable storage medium. Similarly, systems consistent with the present disclosure may include at least one processor and memory, and the memory may be a tangible non-transitory computer-readable storage medium. As used herein, a tangible non-transitory computer-readable storage medium refers to any type of physical memory on which information or data readable by at least one processor may be stored. Examples include random access memory (RAM), read-only memory (ROM), volatile memory, non-volatile memory, hard drives, CD ROMs, DVDs, flash drives, disks, registers, caches, and any other known physical storage medium. Singular terms, such as “memory” and “computer-readable storage medium,” may additionally refer to multiple structures, such a plurality of memories or computer-readable storage media. As referred to herein, a “memory” may comprise any type of computer-readable storage medium unless otherwise specified. A computer-readable storage medium may store instructions for execution by at least one processor, including instructions for causing the processor to perform steps or stages consistent with embodiments herein. Additionally, one or more computer-readable storage media may be utilized in implementing a computer-implemented method. The term “non-transitory computer-readable storage medium” should be understood to include tangible items and exclude carrier waves and transient signals.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure. Therefore, it is intended that the disclosed embodiments and examples be considered as examples only, with a true scope of the present disclosure being indicated by the following claims and their equivalents.

The embodiments may further be described using the following clauses:

1. A system, comprising: at least one processor; and at least one computer-readable, non-transitory medium containing instructions that, when executed by the at least one processor, cause the system to perform operations comprising: obtaining training data for first subjects satisfying a cognitive impairment condition, the training data comprising, for each first subject: baseline data for the first subject, the baseline data comprising baseline cognitive data and image data comprising one or more brain region measurements for one or more brain regions identified as hubs or one or more composite values for one or more clusters of brain regions identified as modules, the one or more hubs or one or more modules identified using network analysis or multi-level clustering; and cognitive impairment progression data for the first subject, the cognitive impairment progression data including repeated measurements acquired over time, the repeated measurements acquired after the baseline cognitive data; training a predictive model, using the training data, to predict cognitive impairment progression data for a first subject using baseline data for the first subject; obtaining the baseline data for a second subject, the second subject satisfying the cognitive impairment condition; and predicting cognitive impairment progression data for the second subject by inputting the baseline data for the second subject to the trained predictive model.

2. The system of clause 1, wherein the repeated measurements include or depend upon: a clinical dementia sum of boxes (CDR-SB) measurement; an Alzheimer's Disease Composite Score (ADCOMS) measurement; or an Alzheimer's Disease Assessment Scale (ADAS) measurement.

3. The system of any one of clauses 1-2, wherein the one or more brain region measurements for the one or more hubs or the one or more composite values for the one or more modules are derived from MRI, CT, or PET images.

4. The system of any one of clauses 1-3, wherein the one or more brain region measurements for the one or more hubs or the one or more composite values for the one or more modules are derived from MRI images.

5. The system of any one of clauses 1-4, wherein the one or more hubs or the one or more modules are identified using network analysis or multi-level clustering.

6. The system of any one of clauses 1-5, wherein the one or more hubs or the one or more modules are identified by: generating a planar-filtered network graph including nodes corresponding to brain regions and edges corresponding to correlations between brain region measurements for the brain regions; generating a hierarchy of network modules by iteratively clustering the nodes into the network modules using the planar-filtered network graph, the hierarchy of the network modules including the modules; and identifying nodes as hubs using within-cluster connectivity between the nodes, the hubs including the hubs.

7. The system of any one of clauses 1-6, wherein the one or more hubs or the one or more modules are identified using multiscale embedded gene co-expression network analysis (MEGENA).

8. The system of any one of clauses 1-7, wherein the one or more brain region measurements for the one or more brain regions comprises volume, surface area, or thickness measurements.

9. The system of clause 8, wherein the one or more hubs comprise: a first hub comprising a middle temporal cortical region, a second hub comprising an inferior parietal cortical region, a third hub comprising an inferior temporal cortical region; or a fourth hub comprising a superior frontal cortical region.

10. The system of any one of clauses 1-9, wherein the one or more composite values are derived from volume, surface area, or cortical thickness measurements for brain regions within the one or more modules.

11. The system of clause 10, wherein the one or more modules comprise: a first network module comprising an inferior parietal region, an inferior temporal region, a middle temporal region, and cortical areas around superior temporal sulcus; or a second network module comprising entorhinal cortex and temporal pole regions.

12. The system of any one of clauses 1-11, wherein the baseline cognitive data for the first subject comprises at least one of a Cogstate Brief Battery score, an International Shopping List Test score, an ADAS score, a mini-mental state examination (MMSE) score, a CDR-SB score, or a FAQ score.

13. The system of any one of clauses 1-12, wherein the baseline cognitive data for the patient comprises at least one of an ADCRL word recall score, an ADCIP ideational praxis score, an ADCRG word recognition score, an ADCDIF word finding difficulty score, a CDR0106 personal care score, an ADCCP constructional praxis score, an ADCNC number cancellation score, an ADCOR orientation score, a CDR0102 orientation score, a CDR0103 judgment and problem solving score, or an ADCDRL delayed word recall score.

14. The system of any one of clauses 1-13, wherein the baseline data for the first subject further comprises demographic data comprising age, sex, or BMI for the first subject.

15. The system of any one of clauses 1-14, wherein the baseline data for the first subject further comprises genomic data.

16. The system of clause 15, wherein the genomic data comprises an ApoE4 allelic count.

17. The system of any one of clauses 1-16, wherein the baseline data for the first subject further comprises plasma, serum, or cerebrospinal fluid biomarker data.

18. The system of clause 17, wherein the plasma, serum, or cerebrospinal fluid biomarker data for the first subject further comprises one or more of: one or more of a cerebrospinal fluid Aβ1-42 score, cerebrospinal fluid Aβ1-40 score, cerebrospinal combined fluid Aβ1-42 and fluid Aβ1-40 score, cerebrospinal fluid ratio of Aβ1-42 to Aβ1-40 score, cerebrospinal fluid total tau score, cerebrospinal fluid neurogranin score, cerebrospinal fluid neurofilament light (NfL) peptide score, or cerebrospinal fluid microtubule binding region (MBTR)-tau score, or one or more of a serum or plasma level Aβ1-42 score, serum or plasma level Aβ1-40 score, serum or plasma level combined Aβ1-42 and Aβ1-40 score, serum or plasma level ratio of Aβ1-42 to Aβ1-40 score, serum or plasma level total tau score, serum or plasma level phosphorylated tau score, serum or plasma level glial fibrillary acidic protein (GFAP) score, or serum or plasma level NfL, peptide score.

19. The system of clause 18, wherein: the serum or plasma level phosphorylated tau score comprises a serum or plasma level tau phosphorylated at 181 (p-Tau181) score, a serum or plasma level tau phosphorylated at 217 (p-Tau217) score, or a serum or plasma level tau phosphorylated at 231 (p-Tau231) score.

20. The system of any one of clauses 1-19, wherein the image data further comprises a brain region MRI measurement depending on one or more of a whole brain volume, a cortical thickness, or a total hippocampal volume.

21. The system of any one of clauses 1-20, wherein the image data further comprises one or more of a tau score, an amyloid PET score, or a fluorodeoxyglucose (FDG) PET score.

22. The system of any one of clauses 1-21, wherein satisfaction of the cognitive impairment condition depends on a diagnosis for the first subject of a neurological disease, dysfunction, or injury.

23. The system of clause 22, wherein the neurological disease, dysfunction, or injury comprises Mild Cognitive Impairment, Alzheimer's Disease, or dementia.

24. The system of any one of clauses 1-23, wherein the first subject is amyloid positive.

25. The system of any one of clauses 1-24, wherein: the cognitive impairment progression data for the second subject indicates progression from Mild Cognitive Impairment to Alzheimer's Disease.

26. The system of any one of clauses 1-25, wherein the cognitive impairment progression data for the second subject comprises a CDR-SB change from baseline.

27. The system of clause 26, wherein: the predictive model exhibits a decreasing relationship between the CDR-SB change from baseline and the brain region measurement for a hub of the one or more hubs or the composite value for a module of the one or more modules.

28. The system of clause 27, wherein: the hub comprises a middle temporal cortical region; the hub comprises an inferior parietal cortical region; the module comprises an inferior parietal region, inferior temporal region, middle temporal region, or banks of a superior temporal sulcus region; or the module comprises entorhinal cortex or temporal pole regions.

29. The system of any one of clauses 27-28, wherein: the baseline cognitive data comprises an ADCRL, ADCIP, or ADAS-13 score; and the predictive model exhibits an increasing relationship between the ADCRL, ADCIP, or ADAS-13 score and the CDR-SB change from baseline.

30. The system of any one of clauses 1-29, wherein the operations further include: obtaining trial data for multiple participants in a clinical trial of an Alzheimer's treatment, the multiple participants including the second subject, the trial data comprising baseline data for the multiple participants; predicting cognitive impairment progression data for the multiple participants using the trained predictive model and the baseline data for the multiple participants; and determining an effect of the Alzheimer's treatment using, in part, the cognitive impairment progression data for the multiple participants.

31. The system of any one of clauses 1-29, wherein the operations further include: screening or selecting candidate patients for inclusion in a clinical trial using the trained predictive model.

32. The system of any one of clauses 1-31, wherein the predictive model comprises a tree-based model.

33. The system of clause 32, wherein the tree-based model comprises a gradient boosting model.

34. The system of any one of clauses 1-31, wherein the predictive model comprises: a Bayesian elastic net model; a Bayesian nonlinear regression model; or a neural network model.

35. The system of any one of clauses 1-34, wherein an elapsed time between acquisition of the baseline cognitive data for the first subject and acquisition of a final one of the repeated measurements for the first subject is between 12 and 36 months.

36. The system of clause 35, wherein the elapsed time between acquisition of the baseline cognitive data for the first subject and acquisition of the final one of the repeated measurements for the first subject is between 18 and 24 months.

37. The system of any one of clauses 1-36, wherein the repeated measurements are acquired at time intervals of between 3 and 12 months.

38. A system, comprising: at least one processor; and at least one computer-readable, non-transitory medium containing instructions that, when executed by the at least one processor, cause the system to perform operations comprising: obtaining training data for first subjects satisfying a cognitive impairment condition, the training data comprising, for each first subject: baseline data for the first subject, the baseline data comprising plasma fluid biomarker data; and cognitive impairment progression data for the first subject; training a predictive model, using the training data, to predict cognitive impairment progression data for the first subject using baseline data for the first subject; obtaining baseline data for a second subject, the second subject satisfying the cognitive impairment condition; and predicting cognitive impairment progression data for the second subject by inputting the baseline data for the second subject to the trained predictive model.

39. The system of clause 38, wherein the plasma fluid biomarker is a p-Tau181, Aβ1-42, or Aβ1-40 biomarker.

40. A system, comprising: at least one processor; and at least one computer-readable, non-transitory medium containing instructions that, when executed by the at least one processor, cause the system to perform operations comprising: obtaining training data for first subjects satisfying a cognitive impairment condition, the training data comprising, for each first subject: baseline data for the first subject, the baseline data comprising plasma fluid biomarker data; and brain amyloid data for the first subject; training a predictive model, using the training data, to predict brain amyloid status for the first subject using baseline data for the first subject; obtaining baseline data for a second subject, the second subject satisfying the cognitive impairment condition; and predicting brain amyloid status for the second subject by inputting the baseline data for the second subject to the trained predictive model.

41. The system of clause 40, wherein the plasma fluid biomarker is a p-Tau181, Aβ1-42, or Aβ1-40 biomarker.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A system, comprising:
at least one processor; and
at least one computer-readable, non-transitory medium containing instructions that, when executed by the at least one processor, cause the system to perform operations comprising:
obtaining training data for first subjects satisfying a cognitive impairment condition, the training data comprising, for each first subject:
baseline data for the first subject, the baseline data comprising baseline cognitive data and image data comprising one or more brain region measurements for one or more brain regions identified as hubs or one or more composite values for one or more clusters of brain regions identified as modules, the one or more hubs or one or more modules identified using network analysis or multi-level clustering; and
cognitive impairment progression data for the first subject, the cognitive impairment progression data including repeated measurements acquired over time, the repeated measurements acquired after the baseline cognitive data;
training a predictive model, using the training data, to predict cognitive impairment progression data for a first subject using baseline data for the first subject;
obtaining the baseline data for a second subject, the second subject satisfying the cognitive impairment condition; and
predicting cognitive impairment progression data for the second subject by inputting the baseline data for the second subject to the trained predictive model.

2. The system of claim 1, wherein the repeated measurements include or depend upon:
a clinical dementia sum of boxes (CDR-SB) measurement;
an Alzheimer's Disease Composite Score (ADCOMS) measurement; or
an Alzheimer's Disease Assessment Scale (ADAS) measurement.

3. The system of claim 1, wherein the one or more brain region measurements for the one or more hubs or the one or more composite values for the one or more modules are derived from MRI, CT, or PET images.

4. The system of claim 1, wherein the one or more brain region measurements for the one or more hubs or the one or more composite values for the one or more modules are derived from MRI images.

5. The system of claim 1, wherein the one or more hubs or the one or more modules are identified using network analysis or multi-level clustering.

6. The system of claim 1, wherein the one or more hubs or the one or more modules are identified by:
generating a planar-filtered network graph including nodes corresponding to brain regions and edges corresponding to correlations between brain region measurements for the brain regions;
generating a hierarchy of network modules by iteratively clustering the nodes into the network modules using the planar-filtered network graph, the hierarchy of the network modules including the modules; and
identifying nodes as hubs using within-cluster connectivity between the nodes, the hubs including the hubs.

7. The system of claim 1, wherein the one or more hubs or the one or more modules are identified using multiscale embedded gene co-expression network analysis (MEGENA).

8. The system of claim 1, wherein the one or more brain region measurements for the one or more brain regions comprises volume, surface area, or thickness measurements.

9. The system of claim 8, wherein the one or more hubs comprise:
a first hub comprising a middle temporal cortical region,
a second hub comprising an inferior parietal cortical region,
a third hub comprising an inferior temporal cortical region; or
a fourth hub comprising a superior frontal cortical region.

10. The system of claim 1, wherein the one or more composite values are derived from volume, surface area, or cortical thickness measurements for brain regions within the one or more modules.

11. The system of claim 10, wherein the one or more modules comprise:
a first network module comprising an inferior parietal region, an inferior temporal region, a middle temporal region, and cortical areas around superior temporal sulcus; or
a second network module comprising entorhinal cortex and temporal pole regions.

12. The system of claim 1, wherein the baseline cognitive data for the first subject comprises at least one of a Cogstate Brief Battery score, an International Shopping List Test score, an ADAS score, a mini-mental state examination (MMSE) score, a CDR-SB score, or a FAQ score.

13. The system of claim 1, wherein the baseline cognitive data for the patient comprises at least one of an ADCRL word recall score, an ADCIP ideational praxis score, an ADCRG word recognition score, an ADCDIF word finding difficulty score, a CDR0106 personal care score, an ADCCP constructional praxis score, an ADCNC number cancellation score, an ADCOR orientation score, a CDR0102 orientation score, a CDR0103 judgment and problem solving score, or an ADCDRL delayed word recall score.

14. The system of claim 1, wherein the baseline data for the first subject further comprises demographic data comprising age, sex, or BMI for the first subject.

15. The system of claim 1, wherein the baseline data for the first subject further comprises genomic data.

16. The system of claim 15, wherein the genomic data comprises an ApoE4 allelic count.

17. The system of claim 1, wherein the baseline data for the first subject further comprises plasma, serum, or cerebrospinal fluid biomarker data.

18. The system of claim 17, wherein the plasma, serum, or cerebrospinal fluid biomarker data for the first subject further comprises one or more of:
one or more of a cerebrospinal fluid Aβ1-42 score, cerebrospinal fluid Aβ1-40 score, cerebrospinal combined fluid Aβ1-42 and fluid Aβ1-40 score, cerebrospinal fluid ratio of Aβ1-42 to Aβ1-40 score, cerebrospinal fluid total tau score, cerebrospinal fluid neurogranin score, cerebrospinal fluid neurofilament light (NfL) peptide score, or cerebrospinal fluid microtubule binding region (MBTR)-tau score, or
one or more of a serum or plasma level Aβ1-42 score, serum or plasma level Aβ1-40 score, serum or plasma level combined Aβ1-42 and Aβ1-40 score, serum or plasma level ratio of Aβ1-42 to Aβ1-40 score, serum or plasma level total tau score, serum or plasma level phosphorylated tau score, serum or plasma level glial fibrillary acidic protein (GFAP) score, or serum or plasma level NfL, peptide score.

19. The system of claim 18, wherein:
the serum or plasma level phosphorylated tau score comprises a serum or plasma level tau phosphorylated at 181 (p-Tau181) score, a serum or plasma level tau phosphorylated at 217 (p-Tau217) score, or a serum or plasma level tau phosphorylated at 231 (p-Tau231) score.

20. The system of claim 1, wherein the image data further comprises a brain region MRI measurement depending on one or more of a whole brain volume, a cortical thickness, or a total hippocampal volume.

21. The system of claim 1, wherein the image data further comprises one or more of a tau score, an amyloid PET score, or a fluorodeoxyglucose (FDG) PET score.

22. The system of claim 1, wherein satisfaction of the cognitive impairment condition depends on a diagnosis for the first subject of a neurological disease, dysfunction, or injury.

23. The system of claim 22, wherein the neurological disease, dysfunction, or injury comprises Mild Cognitive Impairment, Alzheimer's Disease, or dementia.

24. The system of claim 1, wherein the first subject is amyloid positive.

25. The system of claim 1, wherein:
the cognitive impairment progression data for the second subject indicates progression from Mild Cognitive Impairment to Alzheimer's Disease.

26. The system of claim 1, wherein the cognitive impairment progression data for the second subject comprises a CDR-SB change from baseline.

27. The system of claim 26, wherein:
the predictive model exhibits a decreasing relationship between the CDR-SB change from baseline and the brain region measurement for a hub of the one or more hubs or the composite value for a module of the one or more modules.

28. The system of claim 27, wherein:
the hub comprises a middle temporal cortical region;
the hub comprises an inferior parietal cortical region;
the module comprises an inferior parietal region, inferior temporal region, middle temporal region, or banks of a superior temporal sulcus region; or
the module comprises entorhinal cortex or temporal pole regions.

29. The system of claim 27, wherein:
the baseline cognitive data comprises an ADCRL, ADCIP, or ADAS-13 score; and
the predictive model exhibits an increasing relationship between the ADCRL, ADCIP, or ADAS-13 score and the CDR-SB change from baseline.

30. The system of claim 1, wherein the operations further include:
obtaining trial data for multiple participants in a clinical trial of an Alzheimer's treatment, the multiple participants including the second subject, the trial data comprising baseline data for the multiple participants;
predicting cognitive impairment progression data for the multiple participants using the trained predictive model and the baseline data for the multiple participants; and
determining an effect of the Alzheimer's treatment using, in part, the cognitive impairment progression data for the multiple participants.

31. The system of claim 1, wherein the operations further include:
screening or selecting candidate patients for inclusion in a clinical trial using the trained predictive model.

32. The system of claim 1, wherein the predictive model comprises a tree-based model.

33. The system of claim 32, wherein the tree-based model comprises a gradient boosting model.

34. The system of claim 1, wherein the predictive model comprises:
a Bayesian elastic net model;
a Bayesian nonlinear regression model; or
a neural network model.

35. The system of claim 1, wherein
an elapsed time between acquisition of the baseline cognitive data for the first subject and acquisition of a final one of the repeated measurements for the first subject is between 12 and 36 months.

36. The system of claim 35, wherein the elapsed time between acquisition of the baseline cognitive data for the first subject and acquisition of the final one of the repeated measurements for the first subject is between 18 and 24 months.

37. The system of claim 1, wherein
the repeated measurements are acquired at time intervals of between 3 and 12 months.

38. A system, comprising:
at least one processor; and
at least one computer-readable, non-transitory medium containing instructions that, when executed by the at least one processor, cause the system to perform operations comprising:
obtaining training data for first subjects satisfying a cognitive impairment condition, the training data comprising, for each first subject:
baseline data for the first subject, the baseline data comprising plasma fluid biomarker data; and
cognitive impairment progression data for the first subject;
training a predictive model, using the training data, to predict cognitive impairment progression data for the first subject using baseline data for the first subject;
obtaining baseline data for a second subject, the second subject satisfying the cognitive impairment condition; and
predicting cognitive impairment progression data for the second subject by inputting the baseline data for the second subject to the trained predictive model.

39. The system of claim 38, wherein the plasma fluid biomarker is a p-Tau181, Aβ1-42, or Aβ1-40 biomarker.

40. A system, comprising:
at least one processor; and
at least one computer-readable, non-transitory medium containing instructions that, when executed by the at least one processor, cause the system to perform operations comprising:
obtaining training data for first subjects satisfying a cognitive impairment condition, the training data comprising, for each first subject:
baseline data for the first subject, the baseline data comprising plasma fluid biomarker data; and
brain amyloid data for the first subject;
training a predictive model, using the training data, to predict brain amyloid status for the first subject using baseline data for the first subject;
obtaining baseline data for a second subject, the second subject satisfying the cognitive impairment condition; and
predicting brain amyloid status for the second subject by inputting the baseline data for the second subject to the trained predictive model.

41. The system of claim 40, wherein the plasma fluid biomarker is a p-Tau181, Aβ1-42, or Aβ1-40 biomarker.
